SEQUENCE LISTING (42 sequences)

SEQ ID NO: 1 - DNA, 22 bp, synthetic construct
  source 1..22: mol_type = other DNA
gcctttgccatccaagagatgc

SEQ ID NO: 2 - DNA, 22 bp, synthetic construct
  source 1..22: mol_type = other DNA
acactgtctgctggtggagttc

SEQ ID NO: 3 - DNA, 22 bp, synthetic construct
  source 1..22: mol_type = other DNA
gcaatgacctccatcagcagct

SEQ ID NO: 4 - DNA, 22 bp, synthetic construct
  source 1..22: mol_type = other DNA
gtggaagtatgtcctcacagcc

SEQ ID NO: 5 - DNA, 22 bp, synthetic construct
  source 1..22: mol_type = other DNA
cgggaagacaataactgcaccc

SEQ ID NO: 6 - DNA, 23 bp, synthetic construct
  source 1..23: mol_type = other DNA
cggttagcagtatgttgtccagc

SEQ ID NO: 7 - DNA, 20 bp, synthetic construct
  source 1..20: mol_type = other DNA
acgcagcacttcagaatcac

SEQ ID NO: 8 - DNA, 20 bp, synthetic construct
  source 1..20: mol_type = other DNA
cgcagagtctcgccattatg

SEQ ID NO: 9 - DNA, 22 bp, synthetic construct
  source 1..22: mol_type = other DNA
ctctcaagtaccgactccgcta

SEQ ID NO: 10 - DNA, 22 bp, synthetic construct
  source 1..22: mol_type = other DNA
ctgagctgacacctggatgcaa

SEQ ID NO: 11 - DNA, 23 bp, synthetic construct
  source 1..23: mol_type = other DNA
gaccaaaaggtgatgctggacag

SEQ ID NO: 12 - DNA, 22 bp, synthetic construct
  source 1..22: mol_type = other DNA
caagacctcgtgctccagttag

SEQ ID NO: 13 - DNA, 22 bp, synthetic construct
  source 1..22: mol_type = other DNA
tgctgacagaggcaccactgaa

SEQ ID NO: 14 - DNA, 23 bp, synthetic construct
  source 1..23: mol_type = other DNA
cagttgtacgtccagaggcatag

SEQ ID NO: 15 - DNA, 23 bp, synthetic construct
  source 1..23: mol_type = other DNA
cattgctgacaggatgcagaagg

SEQ ID NO: 16 - DNA, 22 bp, synthetic construct
  source 1..22: mol_type = other DNA
tgctggaaggtggacagtgagg

SEQ ID NO: 17 - DNA, 23 bp, synthetic construct
  source 1..23: mol_type = other DNA
acagatgactctctcactcttct

SEQ ID NO: 18 - DNA, 20 bp, synthetic construct
  source 1..20: mol_type = other DNA
agggtctagggccttgagtt

SEQ ID NO: 19 - DNA, 21 bp, synthetic construct
  source 1..21: mol_type = other DNA
gatggggcaaagagggctaaa

SEQ ID NO: 20 - DNA, 20 bp, synthetic construct
  source 1..20: mol_type = other DNA
ccccttcctgggggtataaa

SEQ ID NO: 21 - DNA, 19 bp, synthetic construct
  source 1..19: mol_type = other DNA
tggactcacctggatggca

SEQ ID NO: 22 - DNA, 22 bp, synthetic construct
  source 1..22: mol_type = other DNA
ttgcccaggaggttacaatgaa

SEQ ID NO: 23 - DNA, 21 bp, synthetic construct
  source 1..21: mol_type = other DNA
actcatcactggctcagcatc

SEQ ID NO: 24 - DNA, 20 bp, synthetic construct
  source 1..20: mol_type = other DNA
cacgcgtacttgctctctgt

SEQ ID NO: 25 - DNA, 20 bp, synthetic construct
  source 1..20: mol_type = other DNA
agaaaatcagccctctcggg

SEQ ID NO: 26 - DNA, 20 bp, synthetic construct
  source 1..20: mol_type = other DNA
cattcacgcactcccaggtc

SEQ ID NO: 27 - DNA, 21 bp, synthetic construct
  source 1..21: mol_type = other DNA
catcagctcagagaggcagtt

SEQ ID NO: 28 - DNA, 20 bp, synthetic construct
  source 1..20: mol_type = other DNA
gatcaggatcaagggggtct

SEQ ID NO: 29 - DNA, 22 bp, synthetic construct
  source 1..22: mol_type = other DNA
cgggaagacaataactgcaccc

SEQ ID NO: 30 - DNA, 23 bp, synthetic construct
  source 1..23: mol_type = other DNA
cggttagcagtatgttgtccagc

SEQ ID NO: 31 - DNA, 22 bp, synthetic construct
  source 1..22: mol_type = other DNA
acgagagttgcctggctactag

SEQ ID NO: 32 - DNA, 23 bp, synthetic construct
  source 1..23: mol_type = other DNA
cctcatagatgctaccaaggcac

SEQ ID NO: 33 - DNA, 20 bp, synthetic construct
  source 1..20: mol_type = other DNA
ggctgtattcccctccatcg

SEQ ID NO: 34 - DNA, 22 bp, synthetic construct
  source 1..22: mol_type = other DNA
ccagttggtaacaatgccatgt

SEQ ID NO: 35 - DNA, 20 bp, synthetic construct
  source 1..20: mol_type = other DNA
gcctaacatgcttcgagatc

SEQ ID NO: 36 - DNA, 20 bp, synthetic construct
  source 1..20: mol_type = other DNA
tgatgtctgggtcttggttc

SEQ ID NO: 37 - DNA, 18 bp, synthetic construct
  source 1..18: mol_type = other DNA
gatgagctgatgcaggcc

SEQ ID NO: 38 - DNA, 24 bp, synthetic construct
  source 1..24: mol_type = other DNA
agtcctccacctcgttgtccgtga

SEQ ID NO: 39 - DNA, 18 bp, synthetic construct
  source 1..18: mol_type = other DNA
gcagacgccaacgtccac

SEQ ID NO: 40 - DNA, 20 bp, synthetic construct
  source 1..20: mol_type = other DNA
ccagtcactcagttccccgt

SEQ ID NO: 41 - DNA, 25 bp, synthetic construct
  source 1..25: mol_type = other DNA
cctgctggattacatcaaagcactg

SEQ ID NO: 42 - DNA, 21 bp, synthetic construct
  source 1..21: mol_type = other DNA
tccaacacttcgtggggtcct

(19) United States
(12) Patent Application Publication
(10) Pub. No.: US 2026/0315511 A1
(43) Pub. Date: Oct. 15, 2026

(54) COMPOSITIONS AND METHODS FOR TREATING CANCER

(71) Applicant: The University of North Carolina at Chapel Hill, Chapel Hill, NC (US)

(72) Inventors: Sirui Li, Shanghai (CN); Bhalchandra Mirlekar, Memphis, TN (US); Brandon M. Johnson, Durham, NC (US); Yuliya Pylayeva-Gupta, Chapel Hill, NC (US); Jenny P.-Y. Ting, Chapel Hill, NC (US)

(21) Appl. No.: 19/112,809

(22) PCT Filed: Sep. 20, 2023
(86) PCT No.: PCT/US2023/074642

§ 371 Date: Mar. 18, 2025

Related U.S. Application Data
(60) Provisional application No. 63/376,539, filed on Sep. 21, 2022.
(60) Provisional application No. 63/501,263, filed on May 10, 2023.

(51) Int. Cl. A61K 31/7084; A61K 31/352; A61K 31/381; A61K 31/5377; A61K 31/5415; A61K 39/00; A61K 45/06; A61P 35/00; A61P 37/02; C07K 16/24

(52) U.S. Cl. CPC A61K 31/7084; A61K 31/352; A61K 31/381; A61K 31/5377; A61K 31/5415; A61K 45/06; A61P 35/00; A61P 37/02; C07K 16/244; A61K 2039/505; A61K 2039/545; C07K 2317/76

(57) ABSTRACT

This invention relates to methods of treating cancer, enhancing an immune response and/or reducing and/or reversing immunosuppression to cancer in a subject having, suspected to have and/or at risk of cancer. The invention further relates to compositions, combinations, and kits comprising a STING agonist and IL-35 inhibitor as well as use thereof in the methods of the invention.

### STATEMENT OF PRIORITY

This application claims the benefit, under 35 U.S.C. § 119(e), of U.S. Provisional Application No. 63/376,539, filed Sep. 21, 2022, and U.S. Provisional Application No. 63/501,263, filed May 10, 2023, the entire contents of each of which is incorporated by reference herein in its entirety.

### STATEMENT OF GOVERNMENT SUPPORT

This invention was made with government support under Grant Numbers CA232109, AI029564, AI067798, CA230786, CA196510 and AI100625 awarded by the National Institutes of Health. The government has certain rights in the invention.

### STATEMENT REGARDING ELECTRONIC FILING OF A SEQUENCE LISTING

A Sequence Listing in XML format, entitled 5470-939WO_ST26.xml, 37,929 bytes in size, generated on Sep. 20, 2023, and filed herewith, is hereby incorporated by reference in its entirety for its disclosures.

### FIELD OF THE INVENTION

This invention relates to methods of treating cancer, enhancing an immune response and/or reducing and/or reversing immunosuppression to cancer in a subject having, suspected to have and/or at risk of cancer. The invention further relates to compositions, combinations, and kits comprising a STING agonist and IL-35 inhibitor as well as use thereof in the methods of the invention.

### BACKGROUND OF THE INVENTION

Tumors referred to as being cold, characterized by an immunosuppressive tumor microenvironment, pose a major obstacle to immunotherapy. These tumors include, for example, pancreatic cancer, which has a five-year survival rate of only 10% and few treatment options (Hegde & Chen 2020 Immunity 52:17-35). Preclinical studies have shown that activation of the cGAS-STING pathway in innate cells by STING agonists can elicit antigen-specific adaptive immune responses in a manner that is dependent on type I interferon (IFN-I). However, in clinical trials, STING agonist monotherapy has encountered profound tumor resistance.

### SUMMARY OF THE INVENTION

One aspect of the invention provides a method of treating cancer in a subject, comprising administering to the subject a Stimulator of Interferon Genes (STING) agonist and an interleukin (IL)-35 inhibitor, thereby treating the cancer.

A second aspect of the invention provides a method of enhancing an immune response (e.g., an innate immune cell (e.g., NK cell), B cell and/or T cell response) in a subject having, suspected to have, and/or at risk of cancer, comprising administering to the subject a Stimulator of Interferon Genes (STING) agonist and an interleukin (IL)-35 inhibitor, thereby enhancing the immune response to the cancer.

A third aspect of the invention provides a method of reducing and/or reversing immunosuppression to cancer in a subject, comprising administering to the subject a Stimulator of Interferon Genes (STING) agonist and an interleukin (IL)-35 inhibitor, thereby treating the cancer.

A fourth aspect of the invention provides a composition comprising a STING agonist and IL-35 inhibitor.

A fifth aspect of the invention provides a combination comprising a STING agonist and IL-35 inhibitor.

In some embodiments, a composition and/or combination of the invention may be for use in the methods of the invention.

Also provided is a kit comprising a composition and/or combination of the invention, and optional instructions for the use thereof.

These and other aspects of the invention are set forth in more detail in the description of the invention below.

### BRIEF DESCRIPTION OF THE DRAWINGS

FIG. 1 shows (FIG. 1 panel A): workflow of the orthotopic pancreatic tumor model and treatment. WT mice were injected with KPC4662 tumor cells on Day 0 (d0) and i.v. injected with cGAMP or PBS at indicated timepoints. Tumors were harvested as indicated. (FIG. 1 panel B): Tumor weights at indicated timepoints (n=6 for control (Ctrl) at d11 and d17, n=7 for Ctrl at d21, n=7 for cGAMP-treated groups at d11 and d17, n=8 cGAMP-treated group at d21). (FIG. 1 panel C): Representative scatterplots (left) and frequency (right) of intratumoral CD19⁺ B cells among CD45⁺ cells (n=6 for Ctrl d11 and d17, n=7 for both cGAMP-treated groups d11 and d17, n=17 for both Ctrl and cGAMP-treated groups at d21). (FIG. 1 panel D): Tumor weights at d21 from Cd19^{cre} Tmem173^{fl/fl} or Tmem173^{fl/fl} mice bearing KPC2173 (left) or KPC4662 (right) tumors (KPC2173, n=5 Cd19^{cre} Tmem173^{fl/fl mice and n=}7 Tmem173^{fl/fl}; KPC4662, n=10 Cd19^{cre} Tmem173^{fl/fl} and Tmem173^{fl/fl} mice). (FIG. 1 panel E): Tumor growth (left) and survival curves (right) in E0771-bearing Cd19^{cre} Tmem173^{fl/fl}, Cd4^{cre} Tmem173^{fl/fl} or Tmem173^{fl/fl} mice. Data represent two (FIG. 1 panels B-D) or three (FIG. 1 panel E) independent experiments with symbols for individual mice and mean±s.e.m. FIG. 1 panel D shows a representative experiment. Statistical significance was determined by two-way ANOVA (FIG. 1 panel B), unpaired two-tailed Student's t tests (FIG. 1 panels C and D), unpaired two-tailed Student's t test for area under the curve (AUC) and log-rank (Mantel-Cox) test (FIG. 1 panel E). P values indicated; NS, not significant.

FIG. 2 shows how STING activation contributes to the expansion of IL-35⁺ and IL-10⁺ Breg cells. FIG. 2 panel A: Scatterplots of DEGs in cGAMP-treated vs. untreated WT (left) or cGAMP-treated Tmem173^{-/-} B cells (right) (data adjusted P<0.05; red, upregulated genes; blue, downregulated genes). FIG. 2 panel B: Realtime PCR for Il10, Ebi3 and p35 mRNA in B cells; n=4 per group. FIG. 2 panel C: Percent of IL-35⁺ or IL-10⁺ in CD19⁺ splenic B cells by flow cytometry. FIG. 2 panel D: Percent of IL-35⁺ B cells (left), Breg cells (middle) and IL-35⁺ cells among Breg cells (right) stimulated by different STING agonists in WT and Tmem173^{-/-} splenic B cells. FIG. 2 panel E: IL-35⁺ (top) and IL-10⁺ (bottom) cells among CD19⁺ B cells from indicated mice. FIG. 2 panel F: Relative enrichment of IRF3 by ChIP analysis on Ebi3 (left), p35 (middle) and IL-10 (right) promoters in anti-IgM+/−cGAMP treated B cells from WT or Tmem173^{-/-} mice. IRF3 consensus sequences are shown as red rectangles. FIG. 2 panel G: IL-35 (top) and IL-10 (bottom) levels in KPC4662 tumors from samples in FIG. 1 panel A (n=11 per Ctrl group, n=12 per cGAMP group). FIG. 2 panel H: IL-35 (top) and IL-10 (bottom) levels in tumor homogenates at d21 (n=7 WT Ctrl, n=9 WT cGAMP-treated group, n=4 μMT Ctrl and μMT cGAMP-treated groups). FIG. 2 panel I: IL-35 (top) and IL-10 (bottom) levels in tumors from cGAMP-treated KPC4662 (left, n=16) or KPC2173 bearing mice (right, n=12) at d21. FIG. 2 panel J: Correlation of tumor IL-35 (top) and IL-10 (bottom) protein with tumor weights (left) and the number of intratumoral CD8 (column 2), CD4 (column 3) and NK1.1 (right) cells (n=21). Data represent three independent experiments for all panels except for FIG. 2 panel J (two experiments); mean±s.e.m. Statistical significance was determined by unpaired two-tailed Student's t tests (FIG. 2 panels B, F, H, I), Spearman correlation (FIG. 2 panel J) and two-way ANOVA (FIG. 2 panels C, D, E, G). P values indicated; NS, not significant.

FIG. 3 shows IL-35 ablation in B cells may synergize with STING agonist to modulate NK cell activity. FIG. 3 panel A: Workflow of the pancreatic tumor modeling and treatment. B(Ebi3^{-/-}) mice and B(Ebi3^{+/-}) mice were orthotopically injected with KPC4662 or KPC2173 tumor cells and given cGAMP. FIG. 3 panel B: Bone marrow reconstituted chimeric mice with B cell-specific deficiency in Il10 or p35 were orthotopically injected with KPC4662 tumor cells and treated with cGAMP (i.v.) every three days beginning on d8, with tumors harvested on d21. FIG. 3 panel C: Weights of KPC4662 tumors from cGAMP- and PBS-treated B(Ebi3^{+/-}) and B(Ebi3^{-/-}) mice (left) and B(WT), B(Il10^{-/-}) and B(p35^{-/-}) chimeric mice (right) harvested on d21 (n=11 for Ctrl B(Ebi3^{+/-}) and Ctrl B(Ebi3^{-/-}), n=12 for cGAMP-treated B(Ebi3^{+/-}) and cGAMP-treated B(Ebi3^{-/-}), n=12 for Ctrl and cGAMP-treated B(WT), n=13 for Ctrl and cGAMP-treated B(Il10^{-/-}), n=13 for Ctrl and cGAMP-treated B(p35^{-/-})). FIG. 3 panel D: Tumor weights of cGAMP- or PBS-treated B(Ebi3^{+/-}) and B(Ebi3^{-/-}) mice bearing KPC2173 tumor on d21 (n=6 Ctrl B(Ebi3^{+/-}), Ctrl B(Ebi3^{-/-}) and cGAMP-treated B(Ebi3^{+/-}); n=7, cGAMP-treated B(Ebi3^{-/-})). FIG. 3 panel E: Frequency of tumoral CD19⁺IL-35⁺ B cells from panel C. FIG. 3 panel F: Frequency of tumoral CD45⁺CD8⁺ T cells from panel C. FIG. 3 panel G: Frequency of tumoral CD8⁺ T cells expressing GzmB from panel C. FIG. 3 panel H: Frequency of tumoral TNF⁺CD8⁺ T cells from panel C. FIG. 3 panel I: Frequency of tumoral CD45⁺NK1.1⁺ cells from panel C. FIG. 3 panel J: Frequency of tumoral CD45⁺NK1.1⁺ cells from panel D. FIG. 3 panel K: Frequency of NK1.1⁺ cells expressing GzmB from panel C. Data represent a combination of three independent experiments for all panels except for FIG. 3 panels D and J (two experiments), with each symbol representing an individual animal and mean±s.e.m. Statistical significance was determined by unpaired two-tailed Student's t tests. P values indicated; NS, not significant.

FIG. 4 shows Systemic anti-IL-35 synergizes with STING agonist to potentiate NK cell efficacy. FIG. 4 panel A: Workflow of the pancreatic tumor modeling with cGAMP+anti-IL-35 or anti-IL-10 treatments. FIG. 4 panel B: Tumor weights from panel A collected at d21 (n=11 for Ctrl IgG, n=12 for anti-IL-10 and anti-IL-35-treated groups with or without cGAMP). FIG. 4 panel C: Schematic workflow of the LLC tumor modeling and treatment with cGAMP and/or anti-IL-35. Tumor growth (FIG. 4 panel D) and survival (FIG. 4 panel E) in the LLC mouse model from panel C (n=11 for IgG and cGAMP groups, n=12 for anti-IL-35 and dual therapy groups). FIG. 4 panel F: Number of total intratumoral CD45⁺ leukocytes in mice from panel B. FIG. 4 panel G: Frequency of tumoral CD45⁺NK1.1⁺ cells (left) and GzmB⁺NK1.1⁺ cells (right) from panel B. FIG. 4 panel H: Frequency of tumoral CD45⁺CD8⁺ cells (left) and TNF⁺CD8⁺ T cells (right) from panel B. FIG. 4 panel I: Schematic workflow of the pancreatic tumor modeling with cGAMP and anti-IL-35 plus either anti-CD8 or anti-NK1.1 depleting antibody treatments. FIG. 4 panel J: Tumor images (middle) and weights (right) at d21 in mice from panel I (n=8 for IgG and anti-NK1.1 groups, n=7 for anti-CD8 group). FIG. 4 panel K: Effect of IL-35 on NK cell-mediated lysis of K562 cells assessed by the lactate dehydrogenase (LDH) cell death assay (n=3 per group). Data represent a combination of three independent replicates (FIG. 4 panels B and D-H) or a representative experiment (FIG. 4 panels J and K), with mean±s.e.m. Statistical significance was determined by unpaired two-tailed Student's t tests (FIG. 4 panels B, F-H, J and K), unpaired two-tailed Student's t tests for AUC and Log-rank (Mantel-Cox) test (panel E). P values indicated; NS, not significant.

FIG. 5 shows STING agonist therapy may suppress NK cell-mediated anti-tumor responses via IL-35. FIG. 5 panel A: PCA of RNAseq results from intratumoral NK cells from Ctrl and mice (n=3 per group) treated with cGAMP, anti-IL-35 or a combination of cGAMP+anti-IL-35. FIG. 5 panel B: Enrichment of “glycolysis”, “Pi3k, Akt, mTOR signaling” and “mitotic spindle” pathways in tumoral NK cells from cGAMP+anti-IL-35 vs. Ctrl groups by GSEA as noted in FIG. 14 panel A. FIG. 5 panel C: Venn diagram of upregulated DEGs in combination cGAMP+anti-IL-35, cGAMP or anti-IL-35-treated groups vs. control. FIG. 5 panel D: Heatmap showing n=358 upregulated genes noted in panel C in the Venn diagram. FIG. 5 panel E: Top 10 enriched pathway terms in intratumoral NK cells from dual cGAMP+anti-IL-35-treated group in panel D. FIG. 5 panel F: Heatmap showing upregulated Mitotic cell cycle phase transition genes in intratumoral NK cells as noted in panel E. FIG. 5 panel G: Representative microscopy images of tumor sections from indicated groups as described in FIG. 4 panel A. NK1.1⁺ cell (red) and Ki67⁺ marker (green) stainings are shown. (Scale bars, 100 μm.) FIG. 5 panel H: Quantification of the data in panel G; n=15 per group. FIG. 5 panel I: Realtime-PCR of EBI3, IL12A and IL10 mRNA in human B cells collected from pancreatic cancer patients and healthy donors (n=15, each group) stimulated with anti-IgM+/−cGAMP. HPRT was used as the internal control. The box plots show minima, maxima and median, displaying the 10-90 percentile at the whiskers. FIG. 5 panel J: EBI3, IL12A and IL10 transcript levels in B10, plasmablasts, immature B cells and Br1 cells sorted from peripheral blood of pancreatic cancer patients and healthy donors (n=10 each) and stimulated with anti-IgM+/−cGAMP. For RNAseq, each group has n=3 biological repetitions. Two experiments were combined (FIG. 5 panel H) with mean±s.e.m. Statistical significance was determined by unpaired two-tailed Student's t tests (FIG. 5 panels H-J). P values indicated; NS, not significant.

FIG. 6 shows (FIG. 6 panel A): Representative images of mouse tumors from FIG. 1 panel A. FIG. 6 panel B: Number of tumoral CD45⁺ leukocytes from mice as treated in FIG. 1 panel A (n=6 for Ctrl at d11 and d17, n=7 for cGAMP-treated groups at d11 and d17, n=9 for groups at d21). FIG. 6 panel C: Number of splenic CD45⁺ leukocytes from mice in FIG. 6 panel B. FIG. 6 panel D: Number of tumoral CD19′ B cells in cGAMP or PBS groups from mice in e in FIG. 6 panel B. FIG. 6 panel E: Frequency (left) and number (right) of splenic CD19⁺ B cells among CD45⁺ cells from mice as treated in FIG. 1 panel A (n=6 for Ctrl at d11 and d17, n=7 for cGAMP-treated groups at d11 and d17, n=17 (left) or 9 (right) for d21 groups). FIG. 6 panel F: Frequency of tumoral CD11b⁺ and CD8⁺ cells among CD45⁺ cells (top); frequency and cell number of tumoral CD45⁺CD4⁺ cells (bottom). FIG. 6 panel G: Left, LLC tumor growth in Cd19^{cre} Tmem173^{fl/fl} (n=12), Cd4^{cre} Tmem173^{fl/fl} (n=7) or Tmem173^{fl/fl} (n=9) mice. Right, B16-ova cells were s.c. injected in Cd19^{cre} Tmem173^{fl/fl} (n=8) or Tmem173^{fl/fl} (n=11) mice. Poly(I:C)-ova vaccine was administered at d6. Tumor growth was monitored. FIG. 6 panel H: Realtime PCR revealed Ifnb1 and Ifna4 mRNA levels in KPC4662 at 12 hr post cGAMP treatment (n=3 per group). FIG. 6 panel I: IFN-β in the KPC4662 cell culture supernatants at 24 hr post cGAMP treatment as detected by ELISA. FIG. 6 panel J: Cell viability percentage of KPC4662 at 24 hr post cGAMP treatment as detected by MTT assay (n=3 for Ctrl, n=4 for cGAMP-treated group). Data are the combination of two independent experiments (FIG. 6 panels B-E) or a representative experiment (FIG. 6 panels A and F-J). Mean±s.e.m. is shown, with each symbol representing an individual animal. Unpaired two-tailed Student's t tests was performed for statistical analysis. P values indicated; ns, not significant.

FIG. 7 shows molecular profiles of cGAMP-treated B cells and the effect of STING agonists on B cells. FIG. 7 panel A: Immunoblot analysis of STING pathway components and β-actin in WT and Tmem173^{-/-} B cells stimulated with anti-IgM+/−cGAMP. FIG. 7 panel B: KEGG enrichment of upregulated DEGs in cGAMP-treated WT B cells vs. untreated WT B cells and cGAMP-stimulated Tmem173^{-/-} B cells. FIG. 7 panel C: GSEA analysis of RNAseq data. Normalized Enrichment Score (NES) plot of top10 gene sets enriched in the cGAMP-treated WT B cells. FIG. 7 panel D: GSEA of RNAseq data shows enrichment of “interferon alpha response” (left) and “inflammatory response” pathways (right). FIG. 7 panel E: Heat map of “inflammatory response pathway” genes in cGAMP-treated WT and Tmem173^{-/-} B cells identified increased Ebi3 and Il10 expression (arrowheads). FIG. 7 panel F: Heat map of IL-12 family subunit genes in cGAMP vs. PBS-treated WT and Tmem173^{-/-} B cells. FIG. 7 panel G: Real-time PCR of Ifna4 and Ifnb1 in B cells (n=4 per group) with Actb as the internal control. FIG. 7 panel H: Representative histograms of EBI3 (top, left) and p35 (top, right) induced by cGAMP and analyzed by flow cytometry. Frequency of IL-35⁺ among CD19⁺ B cells treated with anti-IgM+/−cGAMP (bottom); n=4 per group. Green, fluorescence minus one control (FMO). FIG. 7 panel I: Left, IL-10 in anti-IgM/cGAMP-treated B cell supernatants. CD1d^{hi}CD5⁺CD21^{hi} Bregs among CD19⁺ B cells (middle). IL-35⁺ cells among CD1d^{hi}CD5⁺CD21^{hi} Bregs (right); n=4 per group. FIG. 7 panel J: Representative histograms (top) and gMFI plots (bottom) showing abundance of CD19⁺IL-35⁺ (pink), CD19⁺IL-35⁻ (green) and total live CD19⁺ (dark gray) splenic B cells (top) in response to cGAMP. Light gray shadow represents FMO (n=4 per group). FIG. 7 panel K: Frequency of IL-10⁺ (top) among live CD19⁺ B cells and IL-10⁺ cells among CD1d^{hi}CD5⁺CD21^{hi} Bregs (bottom) treated with four different STING agonists. WT and Tmem173^{-/-} splenic B cells were studied as in FIG. 2 panel D. FIG. 7 panel L: Orthotopic pancreatic tumor model experimental schematic, tumor weight and frequency of tumoral and splenic CD19⁺IL-35⁺ B cells, EBI3 and p35 MFI, CD19⁺IL-35⁻ B cell frequency of splenic CD19⁺ B cells in KPC4662 bearing mice treated with DMXAA or diABZI. FIG. 7 panel M: IL-35⁺ (top) and IL-10⁺ (bottom) cells among CD19+B cells sorted from KPC4662 tumors after anti-IgM+/−cGAMP. FIG. 7 panel N: Representative histograms (top, left) and quantification (top, right) of proliferating intratumoral CFSE⁺ (splenic) or Cell Trace Violet (CTV, peritoneal) labelled B cells. The frequency of Breg cells (bottom, left) and expression of immunosuppressive IL-35 (EBI3) and IL-10 cytokines (bottom, right) by CFSE⁺ splenic or CTV⁺ peritoneal Breg cells. n=9 per group. FIG. 7 panel O: IL-35 (top) and IL-10 (bottom) levels in spleens from samples described in FIG. 1 panel A (n=12 per group). FIG. 7 panel P: IL-35 (top) and IL-10 (bottom) levels in spleens from cGAMP-treated KPC4662 (left) (n=16) or KPC2173 (right) (n=12) bearing mice at d21. Representative of three (FIG. 7 panels G-K) or two (FIG. 7 panels A and L-M) independent experiments or combined two experiments (FIG. 7 panels N-P) is shown. For RNAseq, n=3 replicates/group. Mean±s.e.m. with each symbol representing an individual animal. Unpaired two-tailed Student's t tests, unpaired two-tailed Student's t tests for AUC, one-way ANOVA and two-way ANOVA were performed. P values indicated; ns, not significant.

FIG. 8 shows differential effects of cGAMP on IL-35 and IL-10 induction are compared in B and T cells. FIG. 8 panel A: Representative histograms of EBI3 (left), p35 (middle) and IL-10 (right) with geometric mean fluorescence intensity (gMFI) are shown. B and T cells were sorted from spleens of KPC4662 tumors-bearing or non-tumor bearing mice. B cells were stimulated with anti-IgM. T cells were stimulated with anti-CD3 and anti-CD28. Cells were treated with or without cGAMP and analyzed by flow cytometry analysis at 24 hr post treatment. FIG. 8 panel B: Quantification of the percentages of IL-35⁺ (top) and IL-10⁺ (bottom) cells in live CD19⁺ B cells and CD3⁺ T cells (n=4, representative of two independent experiments). FIG. 8 panel C: T_{reg}(Ebi3^{+/-}) and T_{reg}(Ebi3^{-/-}) mice orthotopically inoculated with KPC4662 cells and treated with cGAMP. Representative images of tumors (left) and tumor weights (right) at d21 are shown (n=8 Ctrl PBS, n=8 Ctrl cGAMP, n=9 T_{reg}(Ebi3^{-/-}) PBS, n=9 T_{reg}(Ebi3^{-/-}) cGAMP). Representative of two replicate studies are shown, with each symbol representing an individual animal and mean±s.e.m. denoted. Statistical tests of two-way ANOVA and unpaired two-tailed Student's t tests were performed. P values indicated; ns, not significant.

FIG. 9 shows immune profiling of pancreatic tumor-bearing B cell-specific IL-35 or IL-10 ablation mice. FIG. 9 panel A: Mixed bone marrow chimeras containing B cell-specific deletion of p35 (B(p35^{-/-})) or Il10 (B(Il10^{-/-})) with WT (B(WT)) controls were generated (top) with expected cell phenotypes shown (bottom). FIG. 9 panel B: Validation of loss of p35 and Il10 in B cells from B(p35^{-/-}) and B(Il10^{-/-}) mice. Genomic DNA was isolated from CD19⁺ B cells, CD3⁺ T cells and CD19⁻, CD3⁻ cells (non-T or B cells). Genomic DNA samples from tails of WT, p35^{-/-} and Il10^{-/-} mice were used as controls. Actb was used as the internal control. FIG. 9 panel C: Number of total tumoral CD45⁺ leukocytes from KPC4662 bearing cGAMP- and control PBS-treated mice as in FIG. 3 panel C. FIG. 9 panel D: Frequency of tumoral IL-35 producing CD4⁺) T cells from mice as in FIG. 3 panel C. FIG. 9 panel E: Frequency of tumoral IL-10 producing CD4⁺ T cells from mice as in FIG. 3 panel C. FIG. 9 panel F: Frequency of tumoral IL-35 producing non-T, B cells from mice as in FIG. 3 panel C. FIG. 9 panel G: Frequency of tumoral IL-10 producing non-T, B cells from mice as in FIG. 3 panel C. FIG. 9 panel H: Frequency of tumoral TNF secreting CD4⁺ T cells from mice as in FIG. 3 panel C. FIG. 9 panel I: Frequency of tumoral IFNγ secreting CD4⁺) T cells from mice as in FIG. 3 panel C. FIG. 9 panel J: Frequency of tumoral CD4⁺ Foxp3⁺ Treg cells from mice as in FIG. 3 panel C. Samples analyzed in FIG. 9 panels C-J are described in FIG. 3 panels A and B. Representative experiments or a combination of three independent experiments are shown, with each symbol representing an individual animal and mean±s.e.m. denoted. Unpaired two-tailed Student's t tests were performed for statistical analysis. P values indicated; ns, not significant.

FIG. 10 shows anti-IL-35 may synergize with cGAMP to restrain tumor growth and enhance overall survival in the B16-ova melanoma mouse model. FIG. 10 panel A: B16-ova tumor-bearing mice were vaccinated with poly(I:C)-ova on d6, followed by i.v. administration with cGAMP (n=11), i.p. injection of anti-IL-35 (n=12) or IgG (n=11) or cGAMP+anti-IL-35(n=12) on indicated timepoints. Mice were monitored for tumor growth (FIG. 10 panels B and C) and survival (FIG. 10 panel D). A combination of three independent experiments is shown. Mean±s.e.m. is shown. Unpaired two-tailed Student's t tests and Log-rank (Mantel-Cox) test were performed for statistical analyses. P values indicated; ns, not significant.

FIG. 11 shows data plots of immune cells of combination anti-IL-35⁻ plus cGAMP-treated tumors are examined. All samples analyzed were obtained from KPC4662 tumor-bearing mice that were injected with cGAMP or PBS control in combination with anti-IL-10, anti-IL-35 or control IgG as depicted in FIG. 4 panel A. FIG. 11 panel A: Frequency (left) and number (right) of tumoral IL-35 producing CD19′ B cells in FIG. 4 panel B. FIG. 11 panel B: Number of tumoral CD45⁺NK1.1⁺ cells (left) and NK1.1⁺ cells expressing GzmB (right) as noted in FIG. 4 panel B. FIG. 11 panel C: Number of tumoral CD45⁺CD8⁺ cells (left) and CD8⁺ T cells expressing TNF (right) as noted in FIG. 4 panel B. FIG. 11 panel D: Frequency (left) and number (right) of tumoral CD8⁺ T cells expressing GzmB from mice as in FIG. 4 panel B. FIG. 11 panel E: Frequency (left) and number (right) of tumoral CD8⁺ T cells expressing IFNγ from mice as in FIG. 4 panel B. FIG. 11 panel F: Frequency of tumoral CD45⁺CD4⁺CD25⁻ T effector cells from mice as in FIG. 4 panel B. FIG. 11 panel G: Frequency of tumoral CD4⁺ T cells expressing TNF (left) or IFNγ (right) from mice as in FIG. 4 panel B. FIG. 11 panel H: Frequency of tumoral CD4⁺ Foxp3⁺ Treg cells from mice as in FIG. 4 panel B. A combination of three experiments are shown. Mean±s.e.m. is shown. Unpaired two-tailed Student's t tests were performed for statistical analysis. P values indicated; ns, not significant.

FIG. 12 shows images and data plots of immune profiles of combination cGAMP+anti-IL-35 treated tumors are examined. All samples analyzed were obtained from KPC4662 tumor-bearing mice that were injected with cGAMP or PBS in combination with anti-IL-35 or control IgG as depicted in FIG. 4 panel A. FIG. 12 panel A: H&E, immunohistochemistry with the indicated markers and Sirius red staining of sections from groups as described in FIG. 4 panel A (Scale bars, 100 μm.). FIG. 12 panel B: The frequency of cleaved caspase-3⁺ in cytokeratin 8⁺ cells as shown in FIG. 12 panel A, n=15 per group. FIG. 12 panel C: The number of NK1.1⁺ cells as shown in FIG. 12 panel A, n=12 per group. FIG. 12 panel D: The quantification of collagen deposition depicted by Sirius red staining in sections as shown in FIG. 12 panel A, n=15 per group. FIG. 12 panel E: Real-time PCR results revealed the expression of fibrosis markers Acta2 (αSMA) and Col3a1 in tumor homogenates of indicated groups; n=3 per group. Actb was used as the internal control. A combination of two or representative of two independent experiments are shown. Mean±s.e.m. is shown, Unpaired two-tailed Student's t tests was performed for statistical analysis. P values indicated; ns, not significant.

FIG. 13 shows data plots of dual therapy of cGAMP plus anti-IL-35 depends on TRAIL mediated NK cytotoxicity. FIG. 13 panel A: The efficiency of specific antibody depletion of CD8⁺ or NK1.1⁺ cells in spleens from treated mice (n=8 for IgG and anti-NK1.1 groups, n=7 for anti-CD8 group). FIG. 13 panel B: Flow cytometric analysis of TRAIL, shown as MFI, on tumoral NK1.1⁺ cells in response to IL-35 with IL-15 stimulation for 24 hr (n=5 for Ctrl, n=4 for IL-35-stimulated group). FIG. 13 panel C: Experimental schematic: LLC bearing mice received cGAMP (i.v.) and anti-IL-35 (i.p.) with anti-TRAIL (i.t.). FIG. 13 panel D: Tumor growth of mice described in FIG. 13 panel C. FIG. 13 panel E: Survival curve of mice described in FIG. 13 panel C. A representative of two independent experiments is shown for all panels. Mean±s.e.m. is shown. Unpaired two-tailed Student's t tests, unpaired two-tailed Student's t tests for AUC and Log-rank (Mantel-Cox) test were performed for statistical analysis. P values indicated; ns, not significant.

FIG. 14 shows RNAseq analysis of tumoral NK cells from KPC tumor-bearing mice under mono or dual therapy of cGAMP+anti-IL-35. FIG. 14 panel A: GSEA analysis of RNAseq of tumoral NK cells. Normalized Enrichment Score (NES) plot of top 20 hallmark signature gene sets enriched in combination cGAMP+anti-IL-35 vs. Ctrl treated NK cells. FIG. 14 panel B: GSEA showing the enrichment of “interferon alpha response” in tumoral NK cells isolated from mice treated with cGAMP+anti-IL-35 vs. Ctrl NK cells as noted in FIG. 14 panel A. FIG. 14 panel C: Heatmap of “mitotic spindle pathway” genes in NK cells isolated from animals treated with cGAMP+anti-IL-35 vs untreated Ctrl NK cells. FIG. 14 panel D: Venn diagram showing down-regulated DEGs in combination cGAMP+anti-IL-35, cGAMP or anti-IL-35 vs. Ctrl groups.

FIG. 15 shows data plots of human pancreatic cancer Breg signatures are associated with STING signaling or activated NK cell signature. FIG. 15 panel A: Correlations of EBI3 (top), IL12A (P35) (middle) or IL10 (bottom) with TMEM173 expression in TCGA-PAAD patient samples (n=183). FIG. 15 panel B: Real-time PCR assessment of EBI3 (top), IL12A (middle) and IL10 (bottom) transcript levels in B10 cells, plasmablasts, immature B cells and Br1 cells sorted from pancreatic cancer patients and healthy donors (n=10) and stimulated with anti-CD40+/−cGAMP. HPRT was used as the internal control. FIG. 15 panel C: Correlation of Breg signature to ISG signature or activated NK cell index in TCGA PAAD (n=183) patient samples. FIG. 15 panel D: Correlation of Breg signature to ISG signature or activated NK cell index in TCGA LUAD (n=595) patient samples. Spearman correlation and Unpaired two-tailed Student's t tests were performed. The shaded gray region indicates the 95% confidence bands of the fit, as shown in FIG. 15 panels C and D. P values indicated; ns, not significant.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, +5%, +1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

“Amino acid sequence” and terms such as “peptide,” “polypeptide,” and “protein” are used interchangeably herein, and are not meant to limit the amino acid sequence to the complete, native amino acid sequence (i.e., a sequence containing only those amino acids found in the protein as it occurs in nature) associated with the recited protein molecule. The proteins and protein fragments of the presently disclosed subject matter can be produced by recombinant approaches or can be isolated from a naturally occurring source. The protein fragments can be any size, and for example can range in size from four amino acid residues to the entire amino acid sequence minus one amino acid.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

A “nucleic acid,” “nucleic acid molecule,” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). The different components of the designed protein may provide differing and/or combinatorial function. Structural and functional components of the designed protein may be incorporated from differing and/or a plurality of source material. The designed protein may be delivered exogenously to a subject, wherein it would be exogenous in comparison to a corresponding endogenous protein.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

A “therapeutic,” “therapeutic polypeptide,” “therapeutic molecule” and similar terms refer to a polypeptide and/or molecule that can alleviate, reduce, prevent, delay and/or stabilize symptoms that result from an absence or defect in a protein in a cell or subject and/or is a polypeptide and/or molecule that otherwise confers a benefit to a subject, e.g., anti-cancer effects or improvement in transplant survivability or induction of an immune response.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of cancer.

As used herein, the terms “reduce,” “reduces,” “reduction,” “diminish,” “inhibit” and similar terms mean a decrease of at least about 5%, 10%, 15%, 20%, 25%, 35%, 50%, 75%, 80%, 85%, 90%, 95%, 97% or more.

As used herein, the terms “enhance,” “enhances,” “enhancement” and similar terms indicate an increase of at least about 25%, 50%, 75%, 100%, 150%, 200%, 300%, 400%, 500% or more.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

By “substantially retain” a property and/or to maintain a property “substantially the same” as a comparison (e.g., a control), it is meant that at least about 75%, 85%, 90%, 95%, 96%, 97%, 98%, 99% or 100% of the property (e.g., activity or other measurable characteristic) is retained.

As used herein, the terms “subject” and “patient” are used interchangeably herein and refer to both human and nonhuman animals. A subject of this invention can be any subject that is susceptible to a disorder that can benefit by the methods and compositions of the present invention and/or be treated for a disorder by the methods and compositions of the present invention. In some embodiments, the subject of any of the methods of the present invention is a mammal. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. As a further option, the subject can be a laboratory animal and/or an animal model of disease. Preferably, the subject is a human. The subject may be of any gender, any ethnicity and any age.

A “subject in need thereof” or “a subject in need of” is a subject known to have, or is suspected of having or developing or is at risk of having or developing disorder that can be treated by the methods and compositions of the present invention, or would benefit from the delivery of a particle and/or composition including those described herein.

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). It will be appreciated that the actual method and order of administration will vary according to, inter alia, the particular preparation of compound(s) being utilized, and the particular formulation(s) of the one or more other compounds being utilized. The optimal method and order of administration of the compositions of the invention for a given set of conditions can be ascertained by those skilled in the art using conventional techniques and in view of the information set out herein.

The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the instant compounds can be administered at a dose that will produce effective beneficial effects without causing undue harmful or untoward side effects, i.e., the benefits associated with administration outweigh the detrimental effects.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset are substantially less than what would occur in the absence of the present invention.

A “treatment effective” or “effective” amount as used herein is an amount that is sufficient to provide some improvement or benefit to the subject. Alternatively stated, a “treatment effective” or “effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

An “effective amount” or “therapeutically effective amount” refers to an amount of a compound or composition of this invention that is sufficient to produce a desired effect, which can be a therapeutic and/or beneficial effect. In general, a “therapeutically effective amount” or “treatment effective amount” refers to an amount that is a sufficient, but non-toxic, amount of the active ingredient (i.e., particles of this invention) to achieve the desired effect, which, for example, can be a reduction or elimination in the severity and/or frequency of symptoms and/or improvement or remediation of damage, or otherwise prevent, hinder, retard or reverse the progression of a disease or any other undesirable symptom. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in enhanced natural killer (NK) cells and/or NK cell anti-tumor responses (e.g., expanded population of NK cells, enhanced population of infiltrating NK cells, enhanced expression of granzyme B (gzmB+), and/or expanded population of gzmB+NK cells in the tumor microenvironment), whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to antigens the host views as “non-self”, e.g., immunogens by infection or by vaccination, e.g., cancer antigens. Active immunity can be contrasted with passive immunity, which is acquired through the “transfer of preformed substances (antibody, transfer factor, thymic graft, interleukin-2) from an actively immunized host to a non-immune host.” Id.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

As used herein, “immunosuppressive” refers to a function or activity that suppresses one or more aspects of an active immune response. An “immunosuppressive agent” refers to an agent that inhibits or prevents an immune response, e.g., to a foreign material in a subject. Immunosuppressive agents generally act by inhibiting immune cell (e.g., T-cell, B-cell, NK cell, and the like) activation, disrupting proliferation, and/or suppressing inflammation.

The term “biologically active” as used herein means an enzyme or protein having structural, regulatory, or biochemical functions of a naturally occurring molecule.

The terms “antibody” and “immunoglobulin” include antibodies or immunoglobulins of any isotype, fragments of antibodies that retain specific binding to antigen, including but not limited to Fab, Fv, single chain Fv (scFv), Fc, and Fd fragments, chimeric antibodies, humanized antibodies, single-chain antibodies, and fusion proteins including an antigen-binding portion of an antibody and a non-antibody protein. The antibodies can in some embodiments be detectably labeled, e.g., with a radioisotope, an enzyme which generates a detectable product, a fluorescent protein, and the like. The antibodies can in some embodiments be further conjugated to other moieties, such as members of specific binding pairs, e.g., biotin (member of biotin-avidin specific binding pair), and the like. Also encompassed by the terms are Fab′, Fv, F(ab′)₂, and other antibody fragments that retain specific binding to antigen (e.g., any antibody fragment that comprises at least one paratope).

Antibodies can exist in a variety of other forms including, for example, Fv, Fab, and (Fab′)₂, as well as bi-functional (i.e., bi-specific) hybrid antibodies (see e.g., Lanzavecchia et al., 1987) and in single chains (see e.g., Huston et al., 1988 and Bird et al., 1988, each of which is incorporated herein by reference in its entirety). See generally, Hood et al., 1984, and Hunkapiller & Hood, 1986. The phrase “detection molecule” is used herein in its broadest sense to include any molecule that can bind with sufficient specificity to a biomarker to allow for detection of the particular biomarker. To allow for detection can mean to determine the presence or absence of the particular biomarker member and, in some embodiments, can mean to determine the amount of the particular biomarker. Detection molecules can include antibodies, antibody fragments, and nucleic acid sequences.

As used herein, the term “sample” is used in its broadest sense. In one sense, it is meant to include a specimen from a biological source. A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art. For example, biological samples can be obtained from animals (including humans) and encompass fluids (e.g., blood, mucus, urine, saliva), solids, tissues, cells, and gases. In some embodiments, the sample is obtained from a tumor (e.g., tumor stroma) in the subject. The sample may also comprise one or more immune cells, including T cells of the subject, including immune cells (e.g., helper T cells) from the tumor (e.g., tumor stroma) of the subject.

As used herein the term “control” refers to a comparative sample and/or other reference source for a control subject.

“Control subject” as used herein refers to a subject which does not have said condition(s) of the subject in need, e.g., said cancer and/or an illness to which the methods of the present invention disclosed herein may provide beneficial health effects.

As used herein, “Stimulator of Interferon Genes” or “STING” refers to a transmembrane protein which resides in the endoplasmic reticulum and is also known as TMEM173, MITA, ERIS and/or MPYS. STING is a component of the cGAS-STING cytosolic DNA sensing pathway of the innate immune system. While not wishing to be bound to theory, STING activation is believed to occur upon entry of double-stranded DNA into the cytosol of a cell (e.g., a host cell). STING activators (e.g., agonists) include native and synthetic agonists such as, but not limited to, cyclic CMP-AMP (“cGAMP”), G10, diABZI, ADU-S100, and/or MSA-2. Further description of STING and its activities can be found in Corrales and Gajewski, 2015 Clin Cancer Res 21(21):4774-4779; Hopfner and Hornung 2020 Nat Rev Mol Cell Biol 21:501-521; and Decout et al. 2021 Nat Rev Immunol 21:548-569, the disclosures of each of which are incorporate herein by reference.

As used herein, “tumor microenvironment” or “TME” refers to the immediate and adjacent surroundings of a tumor, including but not limited to, the surrounding stromal environment (e.g., stroma cells), vasculature (e.g., blood vessels), immune cells, fibroblasts and extracellular matrix (ECM), as well as any molecules (e.g., proteins or other macromolecules) encompassed therein. Further description of tumor microenvironments can be found in, e.g., Valkenburg et al. 2018 Nat Rev Clin Oncol 15:366-381 and Ho et al. 2020 Nat Rev Clin Oncol 17:527-540, the disclosures of each of which are incorporated herein by reference.

An immunosuppressive tumor microenvironment is a major obstacle in the control of pancreatic and other solid cancers (Evans et al., 2016 JCI Insight 1(14):e88328; Hegde & Chen 2020 Immunity 52:17-35; Lutz et al., 2014 Cancer Immunology Research 2:616-631). Agonists of the stimulator of interferon genes (STING) protein trigger inflammatory innate immune responses to potentially overcome tumor immunosuppression (Cheng et al., 2018 JCI Insight 3(22):e120638). However, tumor resistance to STING monotherapy has emerged in clinical trials and the mechanism(s) are unclear (Amouzegar et al., 2021 Cancers 13(11):2695). The present invention is based, at least in part, on the demonstration that administration of five STING agonists, including cGAMP, results in an expansion of human and mouse IL-35 regulatory B lymphocytes in pancreatic cancer. While not wishing to be bound to theory, mechanistically, cGAMP may drive expression of IL-35 by B cells in an IRF3-dependent but type I interferon-independent manner.

In several preclinical cancer models, the loss of STING signaling in B cells increases tumor control. Anti-IL-35 blockade or genetic ablation of IL-35 in B cells also reduces tumor growth. As described herein, the inventors of the present application show that the STING-IL-35 axis in B cells may reduce proliferation of natural killer (NK) cells and attenuate the NK-driven anti-tumor response. While not wishing to be bound to theory, these findings may indicate an intrinsic barrier to systemic STING agonist monotherapy, and provide a combinatorial strategy to overcome immunosuppression in tumors.

Accordingly, one aspect of the present invention provides a method of treating cancer in a subject, comprising administering to the subject a Stimulator of Interferon Genes (STING) agonist and an interleukin (IL)-35 inhibitor, thereby treating the cancer.

Another aspect of the present invention provides a method of enhancing an immune response (e.g., an innate immune cell (e.g., NK cell), B cell and/or T cell response) in a subject having, suspected to have, and/or at risk of cancer, comprising administering to the subject a Stimulator of Interferon Genes (STING) agonist and an interleukin (IL)-35 inhibitor, thereby enhancing the immune response to the cancer.

Another aspect of the present invention provides a method of reducing and/or reversing immunosuppression to cancer in a subject, comprising administering to the subject a Stimulator of Interferon Genes (STING) agonist and an interleukin (IL)-35 inhibitor, thereby treating the cancer.

In some embodiments, the cancer comprises a tumor microenvironment. In some embodiments, the tumor microenvironment may be immunosuppressive.

In some embodiments, the administering to the subject a STING agonist and an IL-35 inhibitor may enhance natural killer (NK) cells and/or NK cell anti-tumor responses. Examples of NK cell anti-tumor responses include, but not limited to, expanding and/or generating an expanded population of NK cells, expanding and/or generating an enhanced population of infiltrating NK cells, enhancing expression of granzyme B (gzmB+), and/or expanding and/or generating an expanded population of gzmB+NK cells in the tumor microenvironment as compared to a control (e.g., an untreated, singly STING treated and/or IL-35 inhibitor treated subject).

In some embodiments, the subject may have previously received an immunotherapy. In some embodiments, the cancer in the subject may have been resistant to the previous immunotherapy. In some embodiments, the immunotherapy may be an autologous cellular immunotherapy, e.g., chimeric antigen receptor (CAR)-T cell therapy, CAR-NK cell therapy, and/or other modified immune cell (e.g., dendritic cell based therapy, e.g., Sipuleucel-T and the like)). In some embodiments, the immunotherapy may be targeted antibody therapy (e.g., monoclonal antibody therapy) such as, but not limited to, anti-CD20, anti-EGFR, anti-VEGF, anti-VEGFR2, anti-TNFα, anti-CD44, anti-CD19, anti-CD3, anti-EpCAM, anti-IGF1R, anti-MUC1, anti-CD51, anti-integrin, or any other targeted antibody-based therapy with anti-cancer function. In some embodiments, the immunotherapy may be checkpoint inhibitor therapy. In some embodiments, the checkpoint inhibitor therapy may be inhibitors targeting CTLA-4, PD-1 and/or PD-L1. In some embodiments, the checkpoint inhibitor therapy may be, but is not limited to, pembrolizumab, nivolumab, ipilimumab, atezolizumab, avelumab, durvalumab, cemiplimab, amivantamab, apolizumab, bevacizumab, bivatuzumab, blinatumomab, camrelizumab, catumaxomab, cemiplimab, cixutumumab, clivatuzumab (e.g., clivatuzumab tetraxetan) durvalumab, edrecolomab, ertumaxomab, etaracizumab, faricimab, inebilizumab, intetumumab, isatuximab, margetuximab, necitumumab, nimotuzumab, Obinutuzumab, ocrelizumab, ofatumumab, olaratumab, panitumumab, pemtumomab, pertuzumab, racotumomab, ramucirumab, retifanlimab, rituximab, siltuximab, tafasitamab, teclistamab, tisotumab, tositumomab, trastuzumab, tremelimumab, votumumab and/or any variant or biosimilar thereof.

In some embodiments, the administering to the subject a STING agonist and an IL-35 inhibitor may reduce B regulatory cell (“Breg”) cells; e.g., may reduce a Breg population in the tumor microenvironment as compared to a control (e.g., an untreated, singly STING treated and/or IL-35 inhibitor treated subject).

A STING agonist of the present invention may be any molecule which directly interacts (e.g., contacts and/or binds) STING and results in STING activation. In some embodiments, the STING agonist may be, but is not limited to G10, cGAMP, DMXAA, diABZI, ADU-S100, and/or MSA-2.

“Interleukin-35” or “IL-35” as used herein refers to a heterodimeric interleukin protein comprising an IL-12a chain and an IL-27βchain. It is a member of the IL-12 cytokine family, including but not limited to IL-12, IL-23, IL-27 and IL-35. The IL-12 family of heterodimeric interleukins are each composed of two protein chains from a subset of shared chains including but not limited to IL-12α (also known as IL12-p35 or p35), IL-120 (also known as IL12-p40 or p40), EBI3, IL-27α (also known as IL27-p28, p28 or IL-30), IL-23α (also known as IL23-p19 or p19), wherein IL-35 is composed of the chains IL-12α and IL-27β. The family binds to receptors formed from a subset of shared chains including but not limited to IL-12Rβ1, IL-12Rβ2, IL-23R, IL-27Rα and gp130, wherein the IL-35 canonical IL-35 receptor is composed of the chains IL-12Rβ2 and gp130. IL-35 and the IL-12 family of interleukins is further described in Collison and Vignali 2008 Immunological Reviews 226:248-262, the disclosures of which are incorporated herein by reference.

As such, an IL-35 inhibitor of the present invention may be any molecule which directly interacts (e.g., contacts and/or binds) IL-35 and/or its receptor and results in inhibition of IL-35 signaling and/or other function. In some embodiments, the IL-35 inhibitor may be, but is not limited to, an anti-IL-35 antibody or an antigen-binding fragment thereof. In some embodiments, an IL-35 inhibitor may be an anti-IL35 antibody such as but not limited to clone V1.4C4.22.

A cancer relevant to the present invention may be any cancer in which the tumor microenvironment may comprise B regulatory cells (“Bregs”) and/or natural killer cells (“NK cells”). In some embodiments, the cancer may be any cancer such as, but not limited to, pancreatic cancer (e.g., pancreatic adenocarcinoma (PAAD), pancreatic ductal adenocarcinoma (PDAC), and the like), skin cancer (e.g., melanoma, e.g., skin cutaneous melanoma (SKCM) and the like), lung cancer (e.g., Lung adenocarcinoma (LUAD), lung squamous cell carcinoma (LUSC), and the like), mesothelioma (MESO; e.g., pleural mesothelioma, peritoneal mesothelioma, pericardial mesothelioma, testicular mesothelioma, and the like), breast cancer (e.g., breast invasive carcinoma (BRCA) and the like), blood cancer (e.g., lymphoid neoplasm diffuse large B-cell lymphoma (DLBC), acute myeloid leukemia (LAML), chronic myelogenous leukemia (LCML), and the like), adrenocortical carcinoma (ACC), bladder urothelial carcinoma (BLCA), brain cancer (e.g., brain lower grade glioma (LGG), glioblastoma multiforme (GBM), Head and Neck squamous cell carcinoma (HNSC), and the like) cervical squamous cell carcinoma and endocervical adenocarcinoma (CESC), cholangiocarcinoma (CHOL), colon adenocarcinoma (COAD), esophageal carcinoma (ESCA), kidney or liver cancer (e.g., kidney chromophobe (KICH), kidney renal clear cell carcinoma (KIRC), kidney renal papillary cell carcinoma (KIRP), liver hepatocellular carcinoma (LIHC), and the like), ovarian serous cystadenocarcinoma, pheochromocytoma and paraganglioma (PCPG), prostate adenocarcinoma (PRAD), rectum adenocarcinoma (READ), sarcoma (SARC), stomach adenocarcinoma (STAD), testicular germ cell tumors (TGCT), thymoma (THYM), thyroid carcinoma (THCA), uterine carcinosarcoma (UCS), uterine corpus endometrial carcinoma (UCEC), and/or uveal melanoma (UVM). In some embodiments, the cancer may be a solid cancer (e.g., a tumor).

In some embodiments, administering to the subject a STING agonist and an IL-35 inhibitor may comprise one or more administrations (e.g., serial administrations) of the STING agonist and/or the inhibitor of IL-35. For example, in some embodiments, administering to the subject a STING agonist and an IL-35 inhibitor may comprise serial administrations of STING agonist and/or IL-35 inhibitor every 1, 2, 3, 4, 5, 6, 7, 8, 9, 10 or more days (e.g., every 3 days, every 7 days, once a week, twice a week, three times a week etc.), for one or more repeats (e.g., every 3 days repeated for 4 times, twice a week repeated for 3 weeks etc.). In some embodiments, a STING agonist may be administered on one schedule (e.g., every 3 days repeated for 4 times, etc.) together with one or more administrations of IL-35 inhibitor on another schedule (e.g., twice a week for a total of three times, etc.).

In some embodiments, administering to the subject a STING agonist and an IL-35 inhibitor may comprise delivering an effective amount of the STING agonist and/or inhibitor of IL-35. In some embodiments, an effective amount may comprise a concentration of about 1 μg to about 100 ug STING agonist, or any value or range therein, e.g., about 1, 2, 3, 4, 5, 6, 7, 8, 9, 10, 15, 20, 25, 30, 35, 40, 45, 50, 55, 60, 65, 70, 75, 80, 85, 90, 95 or 100 ug STING agonist. In some embodiments, an effective amount may comprise a concentration of about 1 μg to about 500 μg of an IL-35 inhibitor (e.g., an anti-IL35), or any value or range therein, e.g., about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170. 180, 190, 200, 225, 250, 275, 300, 325, 350, 375, 400, 425, 450, 475, or 500 ug of an IL-35 inhibitor. For example, in some embodiments, an effective amount may comprise a concentration of about 1 ug, about 5 ug, about 10 ug, about 30 ug, or about 50 ug STING agonist, and/or about 10 ug, 30 ug, 50 ug, 100 ug, 200 ug, or 300 μg of an IL-35 inhibitor.

In some embodiments, the STING agonist and inhibitor of IL-35 may be concurrently co-administered. In some embodiments, the STING agonist may be administered prior to or after the IL-35 inhibitor.

In some embodiments, the STING agonist and inhibitor of IL-35 may be administered as a single composition.

In some embodiments, the methods of the present invention may further comprise co-delivering a therapeutic agent to the subject.

In some embodiments, the therapeutic agent may be an immunotherapy agent, e.g., checkpoint inhibitor, e.g., anti-PD1, anti-PDL1, anti-CTLA4, and the like.

In some embodiments, the therapeutic agent may be radiation therapy.

In some embodiments, co-delivering the therapeutic agent may comprise concurrent delivery of the therapeutic agent with the administration of the STING agonist and inhibitor of IL-35. In some embodiments, co-delivering the therapeutic agent may comprise delivery of the therapeutic agent prior to the administration of the STING agonist and inhibitor of IL-35 (e.g., about 1 to about 24 hours prior to, about 1 to about 7 days prior to, about 1 to about 4 weeks prior to the administration of the STING agonist and inhibitor of IL-35). In some embodiments, co-delivering the therapeutic agent may comprise delivery of the therapeutic agent after the administration of the STING agonist and inhibitor of IL-35 (e.g., about 1 to about 24 hours after, about 1 to about 7 days after, about 1 to about 4 weeks after the administration of the STING agonist and inhibitor of IL-35).

The invention also provides immunogenic compositions and combinations for use in the methods of the invention, e.g., immunogenic compositions comprising a STING agonist and/or IL-35 inhibitor of the invention and/or a combination comprising a STING agonist and IL-35 inhibitor of the invention. The composition and/or combination can further comprise a pharmaceutically acceptable carrier.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

Accordingly, another aspect of the present invention provides a composition comprising a STING agonist and IL-35 inhibitor.

Another aspect of the present invention provides a combination comprising a STING agonist and IL-35 inhibitor.

In some embodiments, a combination and/or composition of the present invention may further comprise a pharmaceutical carrier, diluent, and/or adjuvant.

In some embodiments, the present invention provides a pharmaceutical composition comprising a STING agonist and/or IL-35 inhibitor of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions and combinations comprising a STING agonist and/or IL-35 inhibitor of the present invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise a STING agonist and/or IL-35 inhibitor of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition or combination comprising a STING agonist and/or IL-35 inhibitor of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc.

In some embodiments, a pharmaceutical composition or combination comprising a STING agonist and/or IL-35 inhibitor of the present invention, and a pharmaceutically acceptable carrier may further comprise an adjuvant. As used herein, “suitable adjuvant” describes an adjuvant capable of being combined with a STING agonist and/or IL-35 inhibitor of the present invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

Also provided herein is a kit comprising a composition and/or combination of the present invention, and optional instructions for the use thereof.

Kits that include particles of this invention and/or a pharmaceutical composition as described herein are also provided herein. Some kits include particles and/or compositions in a container (e.g., vial or ampule), and may also include instructions for use of the particles and/or composition in the various methods disclosed above. The particles and/or composition can be in various forms, including, for instance, as part of a solution or as a solid (e.g., lyophilized powder). The instructions may include a description of how to prepare (e.g., dissolve or resuspend) the particles in an appropriate fluid and/or how to administer the particles for the treatment of the diseases and disorders described herein.

The kits may also include various other components, such as buffers, salts, complexing metal ions and other agents described above in the section on pharmaceutical compositions. These components may be included with the chimeric protein or may be in separate containers. The kits may also include other therapeutic agents for administration with the chimeric protein. Examples of such agents include, but are not limited to, agents to treat the disorders or conditions described above.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1

Preclinical studies have shown that activation of the cGAS-STING pathway in innate cells by STING agonists can elicit antigen-specific adaptive immune responses in a manner that is dependent on type I interferon (IFN-I). In mice with pancreatic and other types of tumor, the intratumoral injection of STING agonist represents the most efficacious route (Sun et al. 2021 Nature Nanotechnology 16:1260-1270), leading to enhanced tumor-intrinsic IFN-I (Amouzegar et al., 2021 Cancers 13(11):2695), elevated CXCR3-dependent anti-tumor immunity (Vonderhaar et al., 2021 Cell Mol Gastroenterol Hepatol 12:41-58) and increased survival (Jing et al., 2019 J Immunother Cancer 7:115). However, in clinical trials, STING agonist monotherapy has encountered profound tumor resistance.

Emerging studies suggest that STING may promote immune evasion and metastasis through increased PD-L1 expression (Ghaffari et al., 2018 Br J Cancer 119:440-449), T cell death (Wu et al., 2020 Immunity 53:115-126) and/or immunoinhibitory molecules including indoleamine 2,3-dioxygenase (Lemos et al., 2020 J Immunother Cancer 8(2):e001182). However, the precise mechanisms contributing to the lack of efficacy of STING monotherapy remain ill-defined (Amouzegar 2021; Gogoi et al., 2020 Vaccines 8(3):453).

A detailed understanding of B cell function is still emerging (Zhang et al., 2021 Nature 599:471-476; Mirlekar et al. 2018 Cancer Immunology Research 6:1014-1024; Helmink et al., 2020 Nature 577:549-555; Meylan et al., 2022 Immunity 55:527-541; Pylayeva-Gupta et al., 2016 Cancer Discovery 6:247-255). While not wishing to be bound to theory, B cells may induce tumor immunity through tertiary lymphoid structure formation (Helmink 2020; Meylan 2022), antibody production and antigen presentation (Hong et al., 2018 Immunity 49:695-708; Cui et al, 2021 Cell 184:6101-6118). In contrast, B regulatory (Breg) cells may have immunosuppressive properties and contribute to immunological tolerance of cancers.

The below study provides data on the impact of STING activation on the expansion of regulatory B cells which then diminish NK cell-mediated anti-tumor immunity.

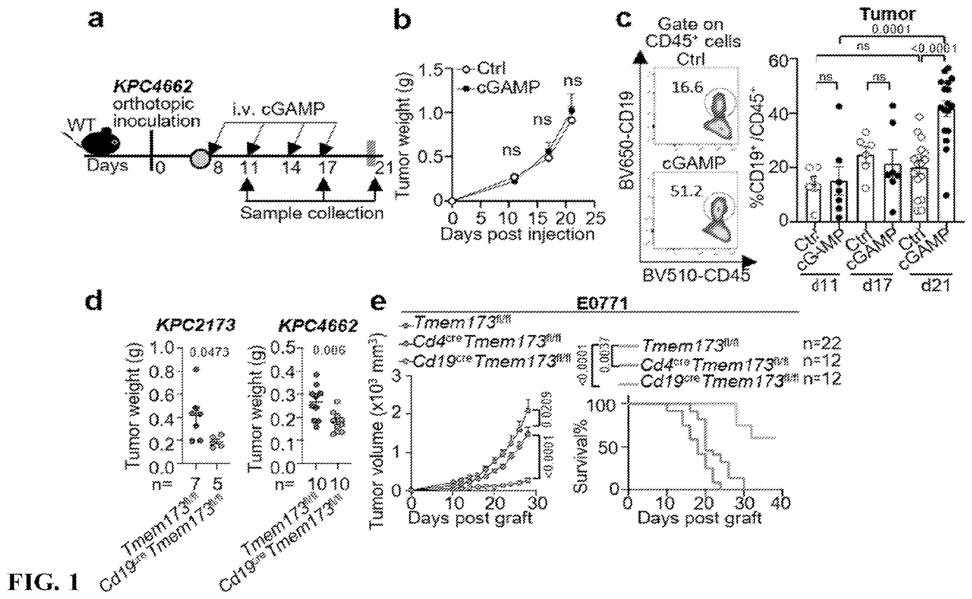

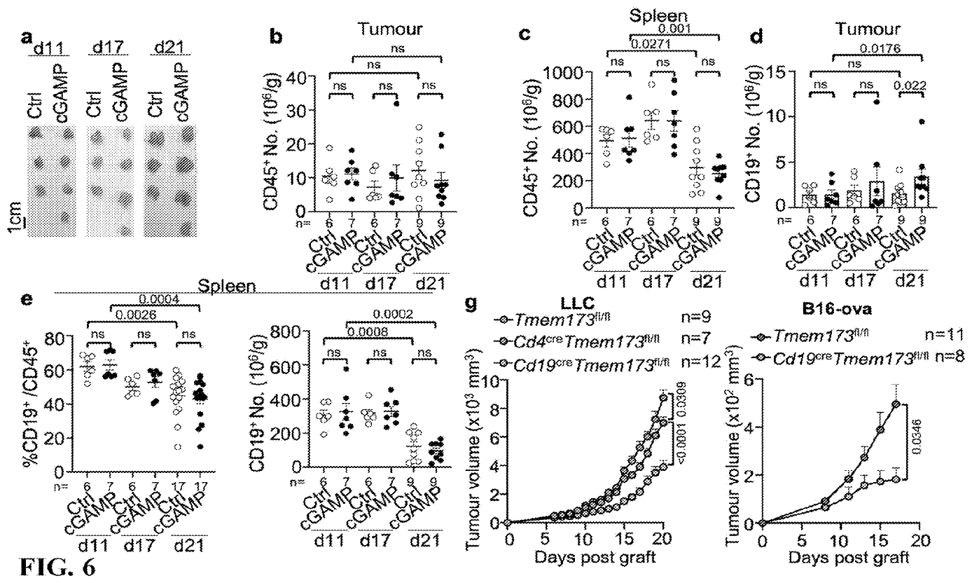

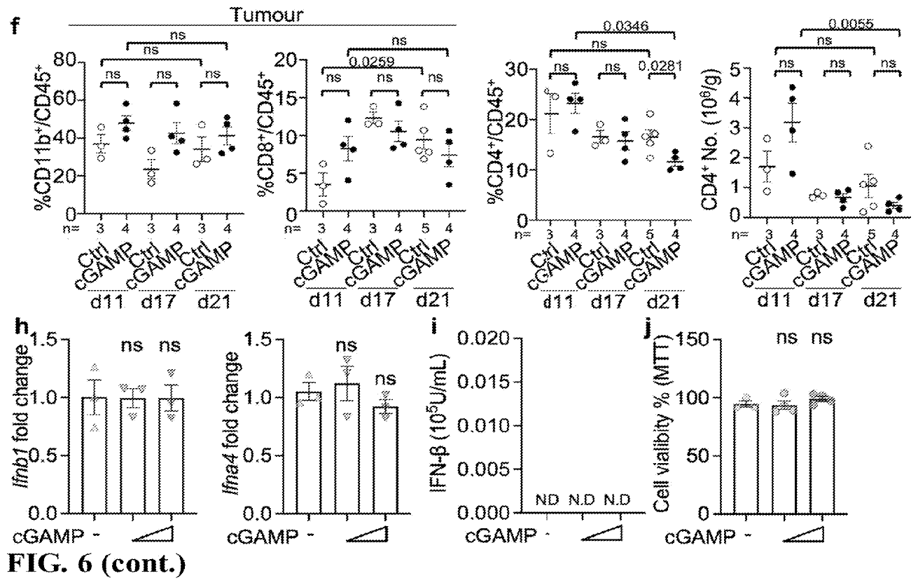

STING activation enhances B cells: To delineate the in vivo response of pancreatic ductal adenocarcinoma (PDAC) to systemic STING agonist exposure, wild-type (WT) mice were injected with mouse PDAC KPC4662 cells derived from LSL-Kras^{G12D/+}; LSL-Trp53^{R172H/+}; Pdx1^{Cre/+} (KPC) mice. After tumors were palpable, mice were given cGAMP (10 μg/mL) intravenously (i.v.) every three days (FIG. 1 panel A) and tumors were collected on days 11, 17 and 21 post-tumor implantation. Administration of cGAMP had no effect on tumor growth (FIG. 1 panel B, FIG. 6 panel A) or CD45⁺ cell number in the tumors or spleens (FIG. 6 panels B and C). However, it increased the percent of CD19⁺ B cells among the CD45⁺cells and the absolute number of B cells in the tumors by day 21 (FIG. 1 panel C, FIG. 6 panel D). In contrast, cGAMP did not alter the percent or number of splenic CD19⁺ cells (FIG. 6 panel E), CD11b⁺ or CD8⁺ cells, and slightly decreased intratumoral CD4⁺ cells (FIG. 6 panel F).

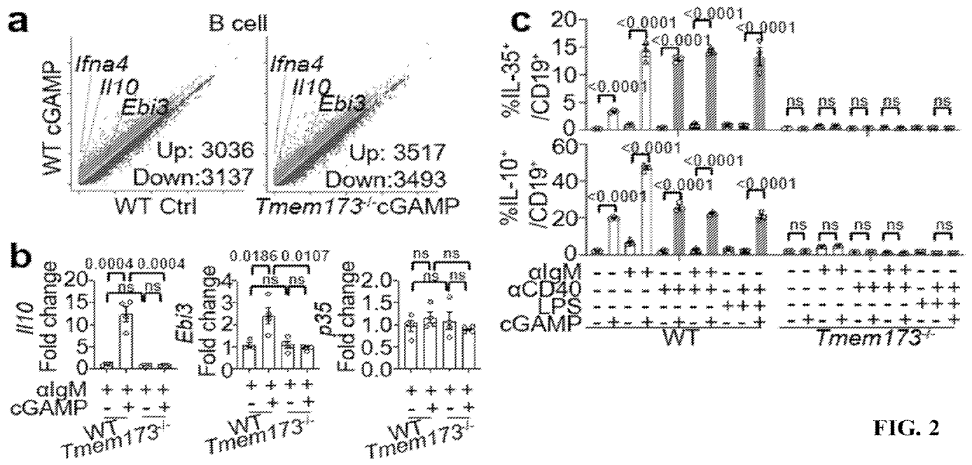

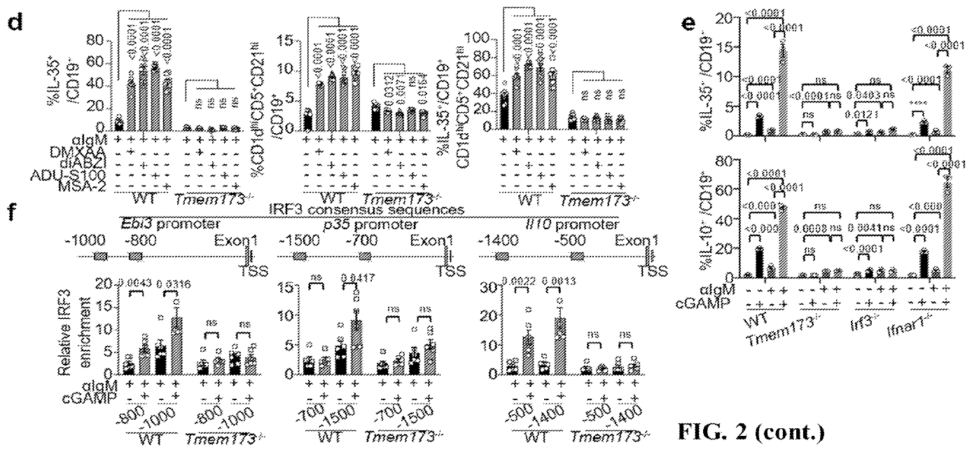

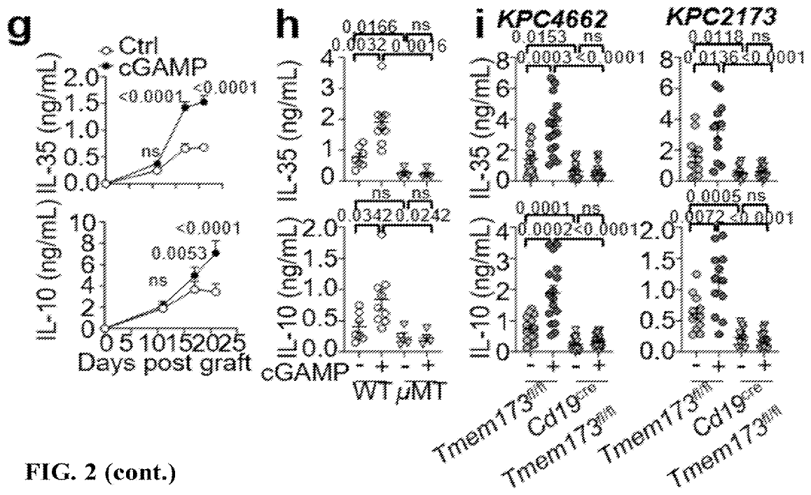

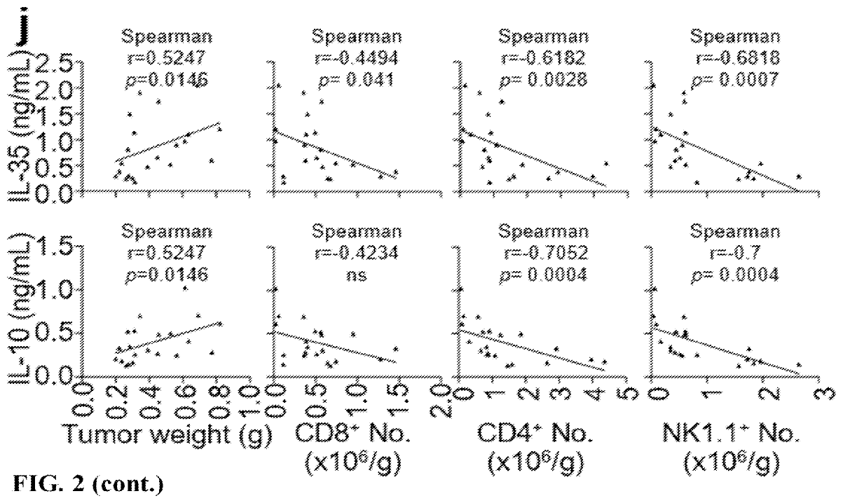

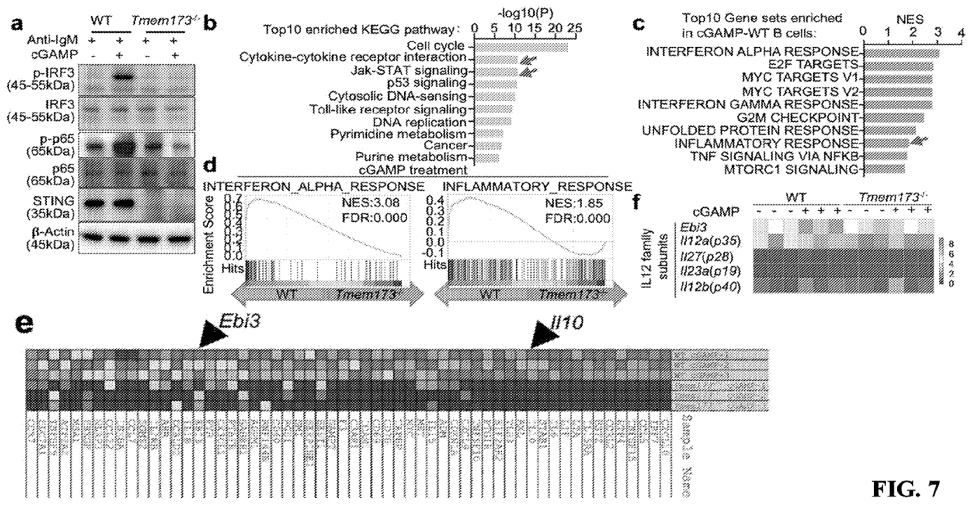

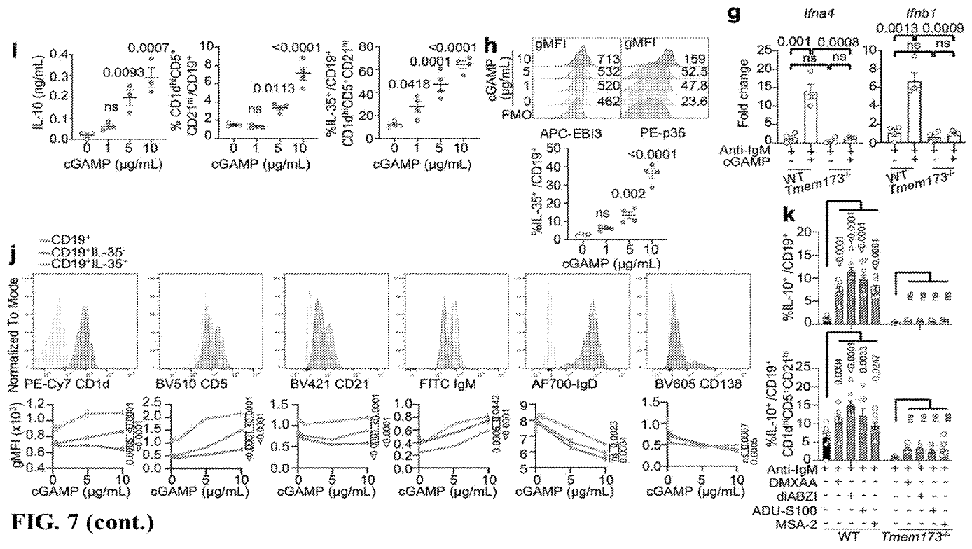

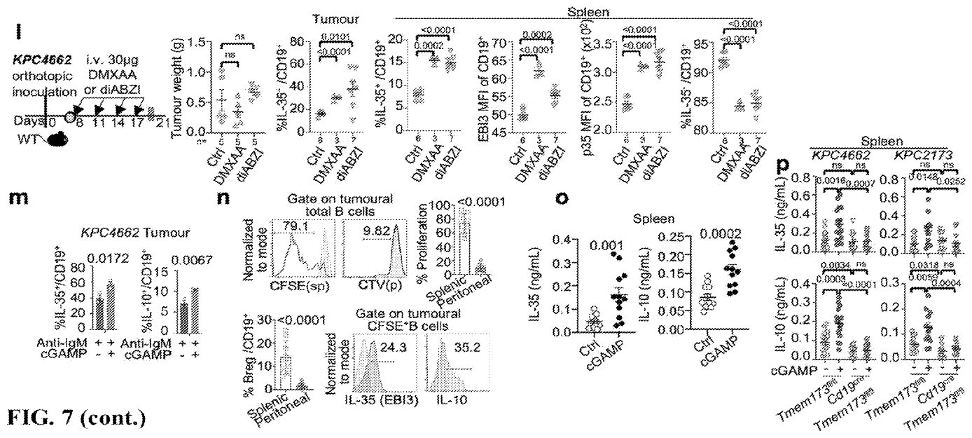

To test the role of STING in B cells, the growth of two orthotopic PDAC tumor lines (KPC2173 and KPC4662) was tested in control mice (Tmem173^{fl/fl}) vs. mice with B cell-specific deletion of the ^{Sting1}, also known as ^{Tmem173} gene (Cd19^{cre}Tmem173^{fl/fl}). In both models, tumor burden was reduced in Cd19^{cre}Tmem173^{fl/fl} mice compared to controls (FIG. 1 panel D). Similar results were obtained for the triple-negative breast cancer cell line E0771 (FIG. 1 panel E), the lung carcinoma (LLC) cell line and the B16-ova melanoma cell line (FIG. 6 panel G). In contrast, E0771 and LLC tumors showed accelerated growth in mice with T cell-specific STING deletion (Cd4^{cre}Tmem173^{fl/fl}) (FIG. 1 panel E; FIG. 6 panel G). In KPC tumor cells, STING appeared to be inactive as cGAMP did not change the expression of Ifnb1/Ifna4 transcripts, IFN-β protein or cell viability (FIG. 6 panels H-J).To explore the effect of cGAMP on B cells, RNA sequencing (RNAseq) was performed on B cells from WT and Tmem173 deficient mice (Tmem173^{-/-}) stimulated using anti-IgM with or without cGAMP. STING activation was verified by increased phospho (p)-IRF3 and p-p65 in WT controls but not in Tmem173^{-/-} B cells (FIG. 7 panel A). Kyoto Encyclopedia of Genes and Genomes (KEGG) pathway enrichment analysis of differentially expressed genes (DEGs) from pairwise comparisons of cGAMP-treated vs. untreated WT B cells and of cGAMP-treated WT vs. Tmem173^{-/-} B cells was conducted. Genes downstream of cytokine-cytokine receptor activation and JAK-STAT signaling pathways were affected by cGAMP as expected (FIG. 7 panel B). Gene set enrichment analysis (GSEA) showed that the top 10 gene sets altered by cGAMP in B cells from WT vs. Tmem173^{-/-} mice, included IFN response and inflammatory response genes (FIG. 7 panels C and D). Surprisingly, Il10 and Ebi3 mRNA were upregulated by cGAMP in 30 WT but not in Tmem173^{-/-} B cells (FIG. 2 panel A, FIG. 7 panel E). EBI3 is a subunit of the immunosuppressive cytokines IL-27 and IL-35, members of the IL-12 family. In B cells, other transcripts of IL-12 family members such as the IL-35 subunit, p35, were unaffected by cGAMP, whereas the expression of IL27, IL23a and IL12b were nearly undetectable (FIG. 7 panel F). These results were confirmed by real-time PCR, which showed that anti-IgM plus cGAMP increased the expression of Ebi3, Il10, Ifna and Ifnb1 in WT, but not in Tmem173−/− B cells, whereas p35 mRNA remained unchanged (FIG. 2 panel B, FIG. 7 panel G).

STING induces B regulatory cells: To confirm cGAMP-induced IL-10 and IL-35, purified splenic B cells from WT and Tmem173^{-/-} mice were treated with anti-CD40, anti-IgM or LPS, with or without cGAMP (FIG. 2 panel C). anti-IgM treatment increased EBI3⁺p35⁺ (IL-35⁺) and IL-10⁺ cells, while anti-CD40 increased IL-35⁺ cells over baseline. cGAMP significantly augmented the frequency of IL-35 and IL-10 in WT but not in Tmem173^{-/-} B cells (FIG. 2C) and the effect was dose-dependent (FIG. 7 panel H). These CD19⁺IL-35⁺ cells were CD1d^{hi}CD5⁺CD21^{h} B cells (FIG. 7 panel J), a phenotype characteristic of murine Breg cells. Compared to anti-IgM alone, cGAMP plus anti-IgM further increased the IL-10 production and the frequency of CD19⁺CD1d^{hi}CD5⁺CD21^{hi} Breg (FIG. 7 panel I, left and middle). Additionally, cGAMP treatment increased the frequency of IL-35⁺ cells among this Breg population (FIG. 7 panel I, right).

Other STING agonists were next tested, including DMXAA, diABZI, ADU-S100 and MSA-2, and all increased the frequency of IL-35⁺ and IL-10⁺ Breg cells (FIG. 2 panel D, FIG. 7 panel K). As observed for cGAMP, DMXAA and diABZI (30 μg) did not reduce tumor growth (FIG. 7 panel I) but increased the percent of CD19⁺IL-35⁺ cells in tumors and spleens, and increased EBI3 and p35 levels in splenic CD19⁺ B cells (FIG. 7 panel I). The frequency of IL-35⁺ or IL-10⁺CD19⁺ cells was also increased by cGAMP in intratumoral B cells (FIG. 7 panel M).

STING-IRF3 mediates Breg cytokines: To delineate the source of B cells that infiltrated PDAC, splenic and peritoneal B cells were labeled with carboxyfluorescein succinimidyl ester (CFSE) and cell trace violet (CTV) respectively, followed by adoptive transfer into B cell-deficient, PDAC-bearing mice treated with cGAMP. The majority of proliferating B cells recovered from tumors were CFSE⁺ (FIG. 7 panel N), indicating a splenic-origin.

To delineate the STING pathways that induced IL-35 and IL-10 expression in B cells, splenic B cells from WT, Tmem173^{-/-}, Irf3^{-/-} or Ifnar1^{-/-} mice were sorted by magnetic-activated cell sorting (MACS), treated with anti-IgM and/or cGAMP ex vivo and analyzed by flow cytometry. Frequencies of IL-35⁺ and IL-10⁺ B cells in response to cGAMP or cGAMP plus anti-IgM were significantly diminished in Tmem173^{-/-} and Irf3^{-/-} but not in Ifnar1^{-/-} B cells (FIG. 2 panel E). Next, we used chromatin immuno-precipitation assays (ChIP) to test for IRF3 binding to the promoter regions (FIG. 2 panel F), and found enhanced binding of IRF3 to the promoters of Ebi3, p35 and Il10 in WT but not in Tmem173^{-/-} B cells (FIG. 2 panel F). Thus, the upregulation of IL-35 and IL-10 expression in CD19⁺ B cells is mediated by an interferon-independent but STING-IRF3-dependent mechanism.

The source of IL-35 and IL-10 in PDAC was next examined. Both cytokines were increased in tumor homogenates as tumors grew and cGAMP significantly elevated the expression of these cytokines in tumors (FIG. 2 panel G) and spleens (FIG. 7 panel O). Intratumoral IL-35 and IL-10 were nearly abrogated in B cell-deficient PMT mice, indicating a B cell-origin for these cytokines (FIG. 2 panel H). IL-35 and IL-10 remained at baseline in Cd19^{cre}Tmem173^{fl/fl} PDAC-bearing mice treated with cGAMP (FIG. 2 panel I, FIG. 7 panel P), indicating that the STING pathway in B cells is required for increased IL-35 and IL-10. To analyze the impact of these immunosuppressive cytokines on tumors, this study showed that increased IL-35 and IL-10 in KPC4662 tumor homogenates positively correlated with tumor weight, and negatively correlated with tumor-infiltrating CD8⁺, CD4⁺ and NK1.1⁺ effectors (FIG. 2 panel J), implicating their contribution to the establishment of an immunosuppressive milieu.

As IL-35 and IL-10 are also expressed by T regulatory cells (Treg), this study explored whether cGAMP enhanced their expression in T cells. T and B cells were MACS-sorted from the spleens of PDAC-bearing and non-tumor-bearing WT mice. B cells were treated with anti-IgM with or without cGAMP and T cells were treated with anti-CD3 and anti-CD28 with or without cGAMP, revealing cGAMP increased IL-35⁺ and IL-10⁺ tumor-associated B cells but not IL-35⁺ or IL-10⁺ T cells (FIG. 8 panels A and B). To assess the role of Treg-derived IL-35, KPC4662 tumors were orthotopically engrafted into mice bearing Treg-specific deletion of Ebi3 (Foxp3^{cre}Ebi3^{fl/fl}) or control Ebi3^{fl/fl} animals. cGAMP treatment caused no difference in tumor weight in these two groups, thus Ebi3 deficiency in Treg cells did not affect tumor growth in mice treated with cGAMP (FIG. 8 panel C).

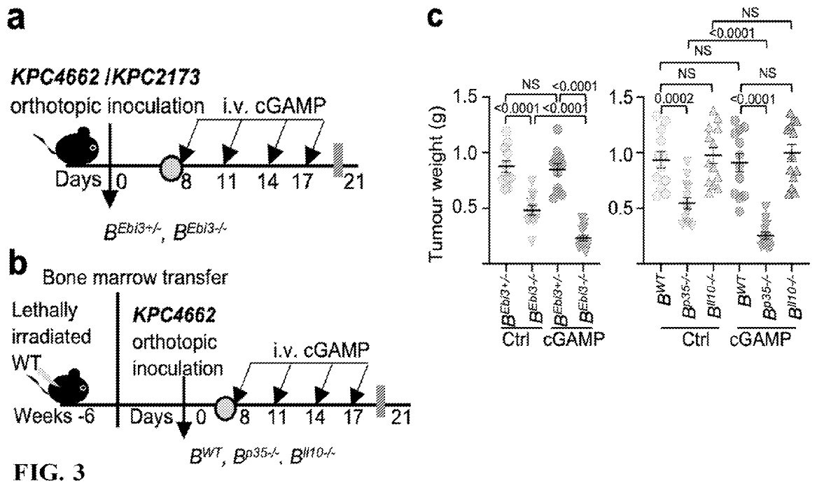

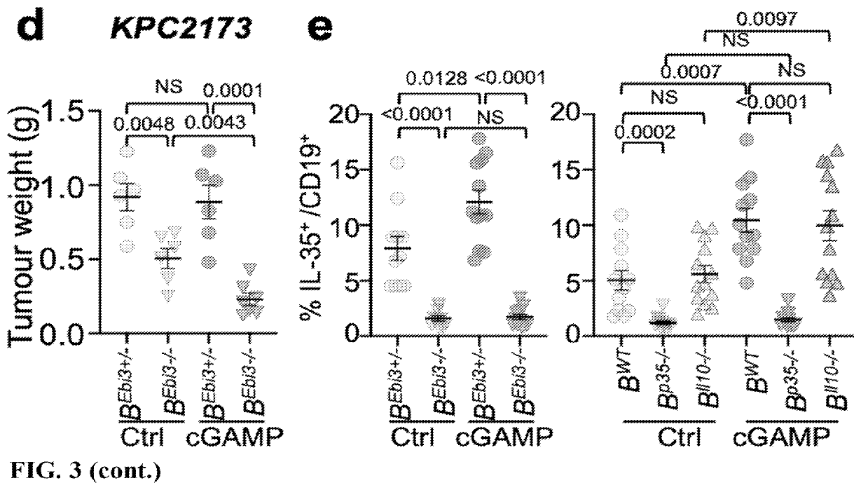

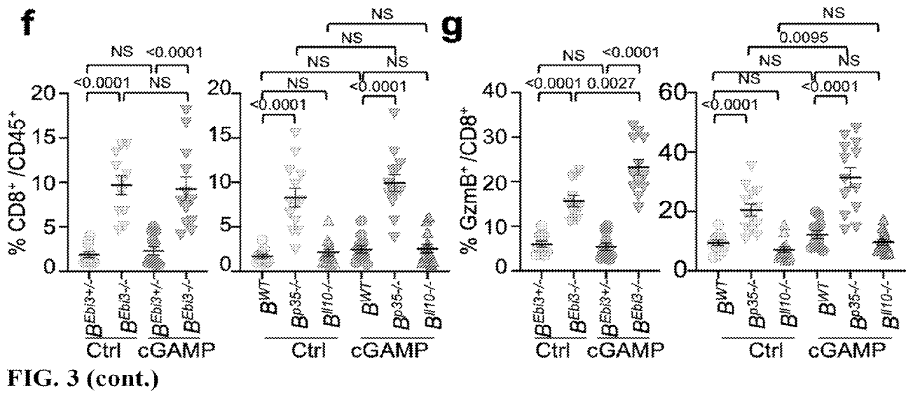

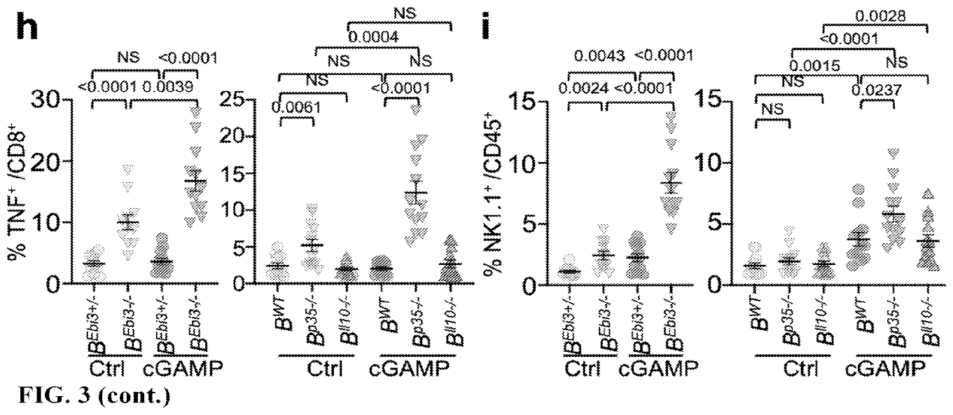

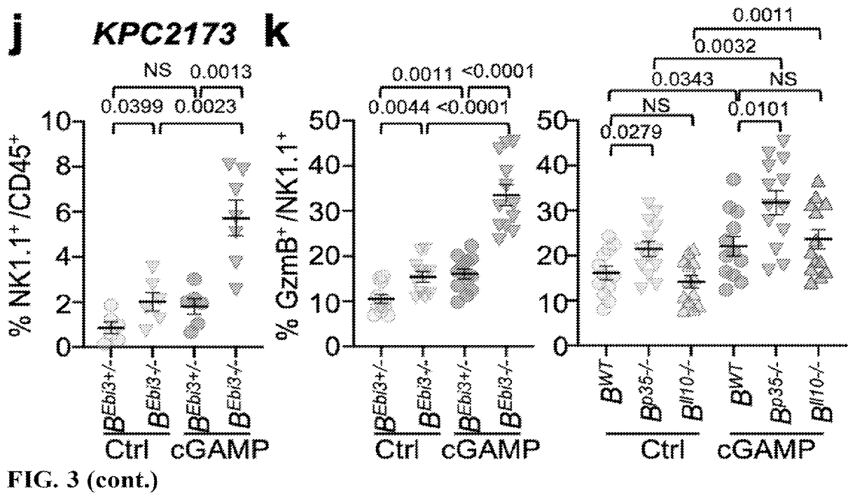

IL-35⁺ B cells subdue NK cell responses: The effect of cGAMP treatment in mice with B cell-specific deficiency in Ebi3, p35 or Il10, designated B(Ebi3^{-/-}) (Cd19^{cre}Ebi3^{fl/fl}), B(p35^{-/-}) and B(Il10^{-/-}), respectively, was next investigated (FIG. 9 panels A and B). These strains were orthotopically injected with KPC cells and treated with PBS or cGAMP (FIG. 3 panels A and B). Treatment with cGAMP reduced tumor growth as compared to PBS in B (Ebi3^{-/-}) and B(p35^{-/-}) mice but not control B(Ebi3^{+/-}) and B(WT) or B(Il10^{-/-}) mice (FIG. 3 panels C and D). Conversely, the treatment increased tumor-infiltrating CD19⁺IL-35⁺ cells in B(Ebi3^{-/-}), B(WT) and B(Il10^{-/-}) but not B(Ebi3^{-/-}) and B(p35^{-/-}) mice (FIG. 6, FIG. 3 panel E). While the lack of Ebi3 and p35 in B cells may increase total CD45⁺CD8⁺ cells in tumor-bearing mice (Mirlekar et al. 2020 Cancer Immunology Research 8:292-308), cGAMP did not further augment this population (FIG. 3 panel F) but instead increased the percent of CD8⁺ cells expressing granzyme (GzmB) or TNF (FIG. 3 panels G and H). Furthermore, B cell-deletion of Ebi3 and p35 modestly increased intratumoral NK1.1⁺ cells, but cGAMP significantly increased NK1.1⁺ cells (FIG. 3 panels I and J). GzmB⁺ NK cells were increased in cGAMP-treated B(Ebi3^{-/-}) and B(p35^{-/-}) mice compared to controls (B(Ebi3^{-/-}), B(WT)) and B(Il10^{-/-}) mice, suggesting that IL-35 but not IL-10 reduced GzmB+NK cells (FIG. 3 panel K). In contrast, total CD45⁺ cells (FIG. 9 panel C), IL-35 or IL-10 expressing CD4⁺ T cells (FIG. 9 panels D and E), cells other than T and B cells were not increased (FIG. 9 panels F and G). B(p35^{-/-}) and B(Ebi3^{-/-}) mice had fewer CD4⁺ Foxp3⁺ Treg cells and increased intratumoral CD4⁺ IFNγ⁺ and CD4⁺ TNF⁺ T cells compared to controls, but these populations were not affected by cGAMP (FIG. 9 panels H-J). Thus, the notable consequence of B cell-specific IL-35 expression is reduced frequency of NK cells.

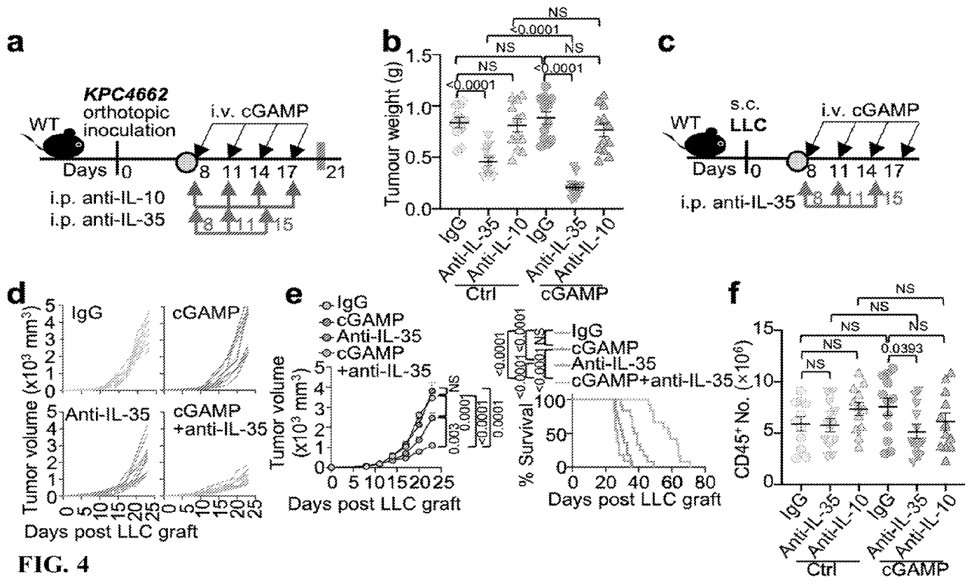

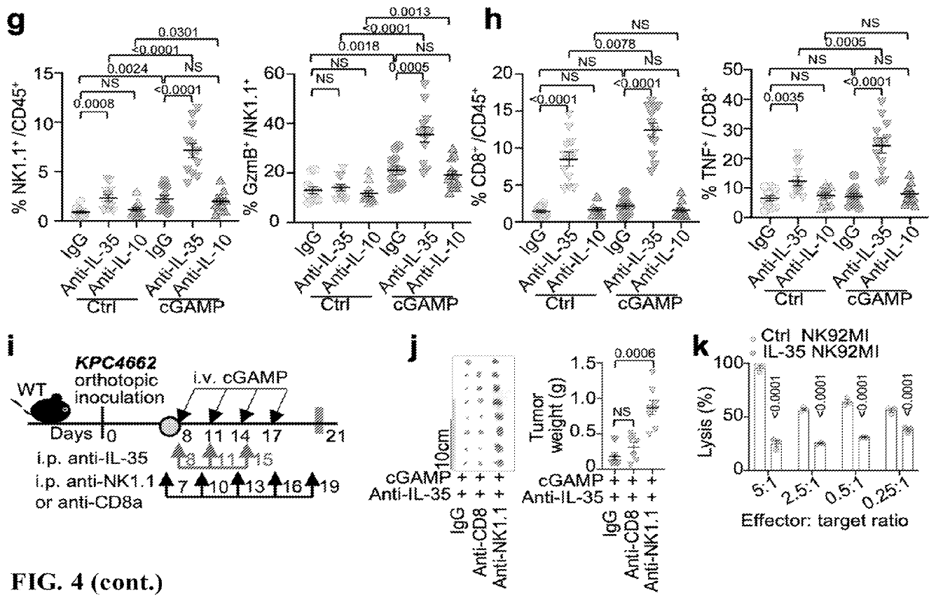

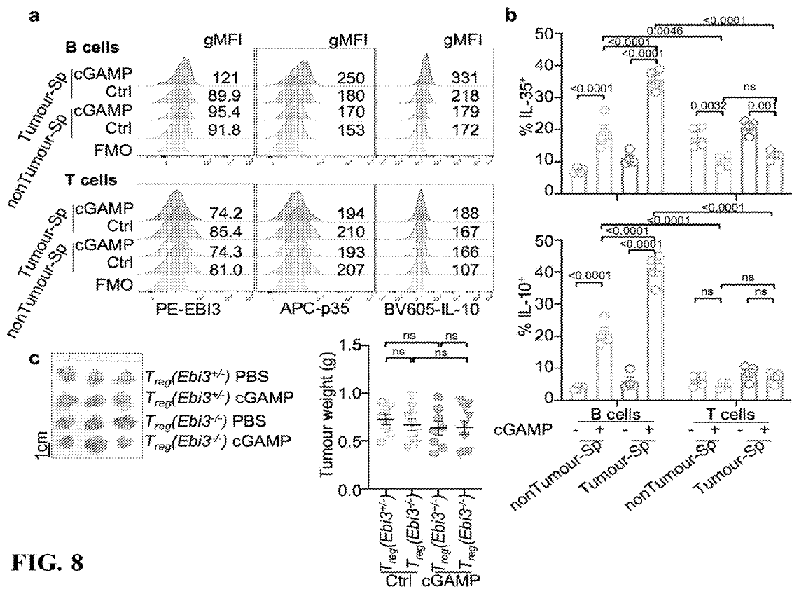

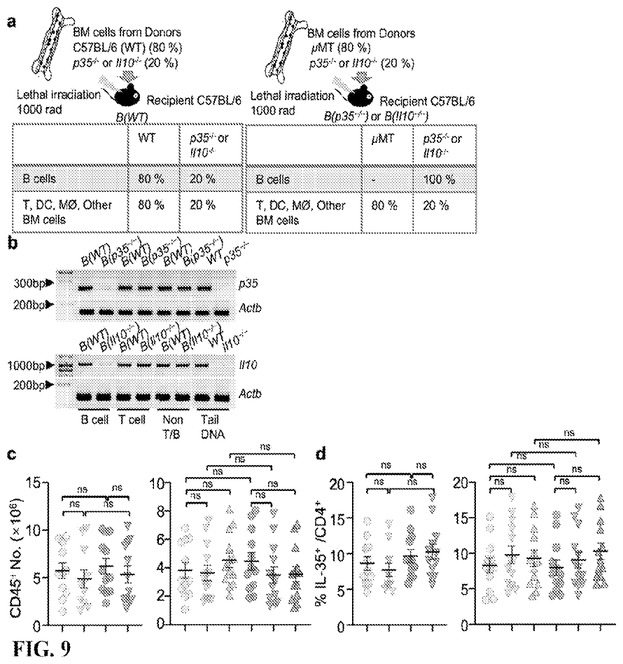

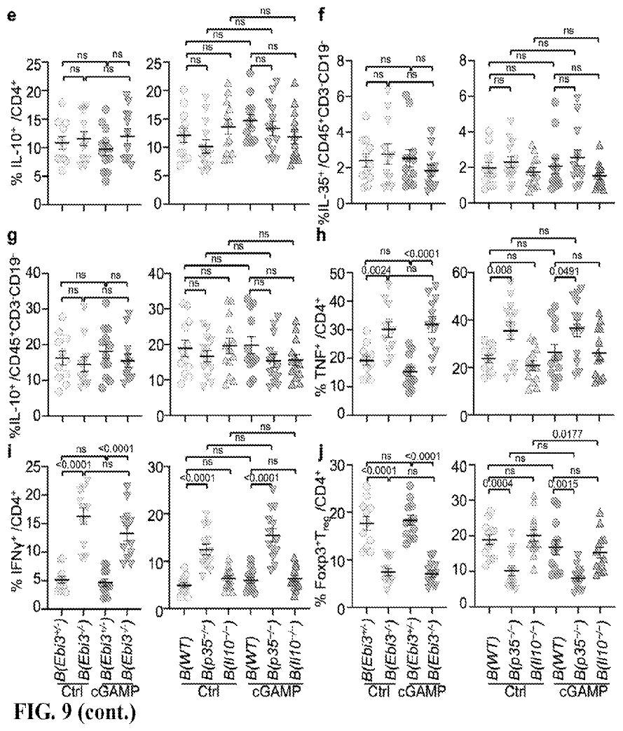

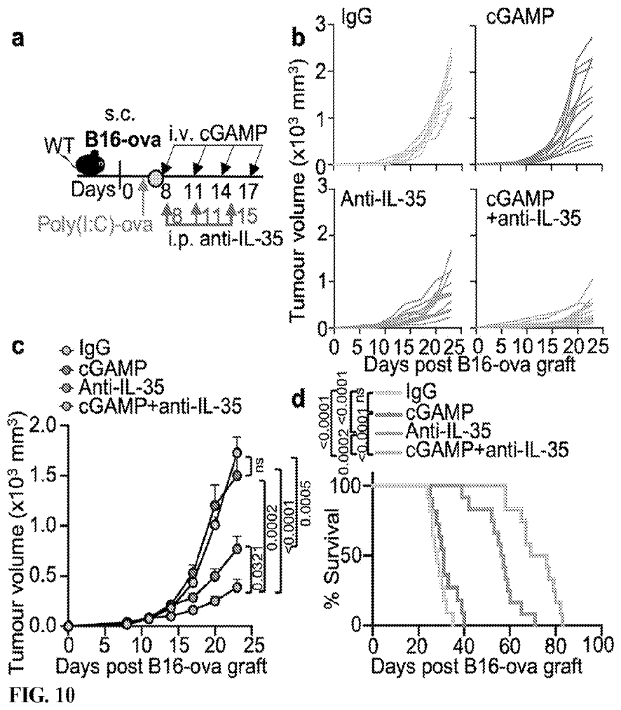

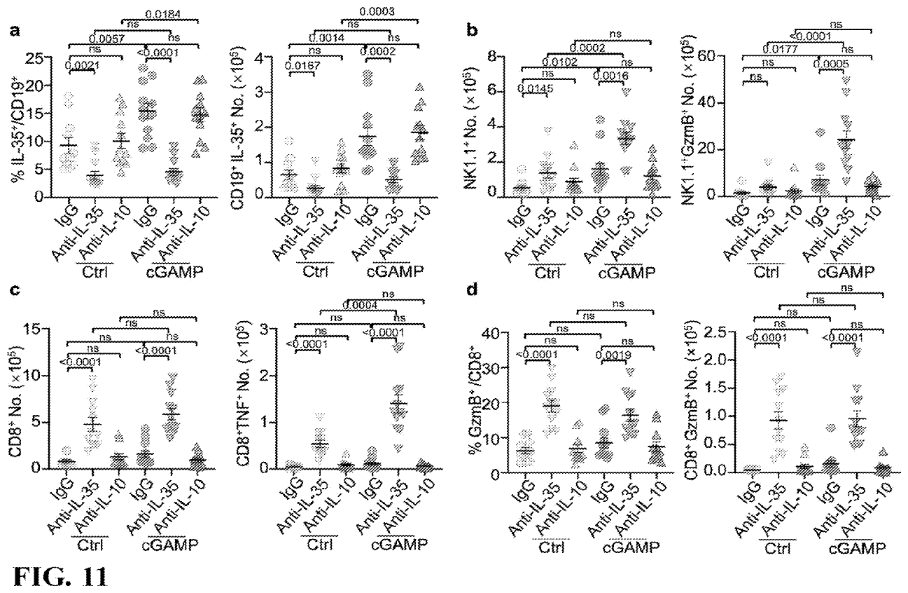

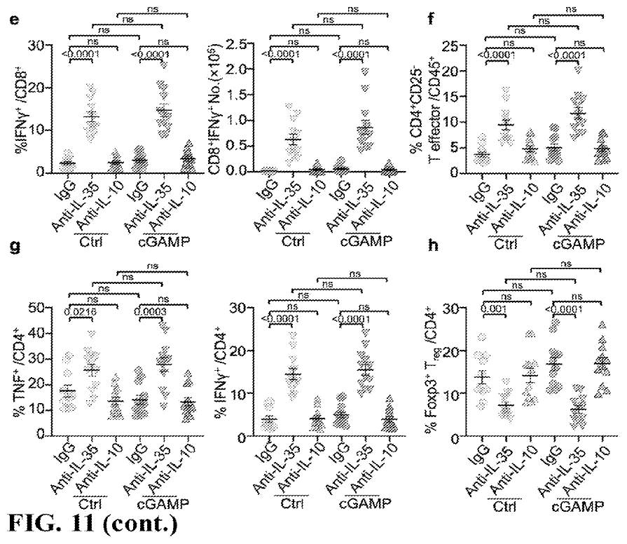

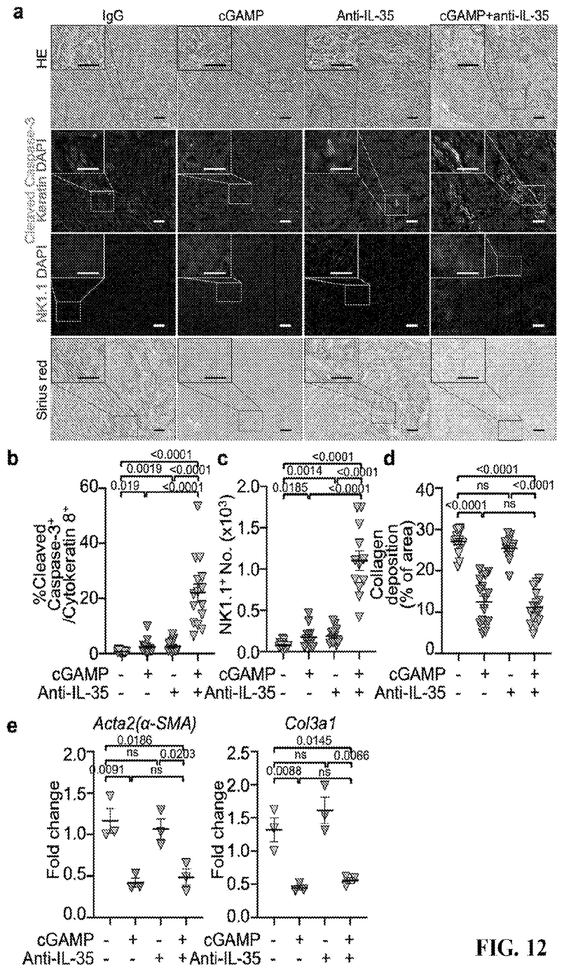

It was next evaluated if cGAMP combined with IL-35 or IL-10 neutralizing antibodies could control the growth of PDAC, Lewis lung cancer (LLC) or B16-ova melanoma (FIG. 4 panels A-E and FIG. 10). Anti-IL-35 combined with cGAMP was most effective in reducing tumor burden and/or prolonging survival. Treatment with anti-IL-35 but not anti-IL-10, reduced the number of intratumoral CD19⁺IL-35⁺ B cells compared to the control group as expected (FIG. 11 panel A). The combination of cGAMP plus anti-IL-35 did not change the number of total CD45⁺ leukocytes (FIG. 4 panel F), but significantly increased infiltrating NK1.1⁺ cells and NK1.1⁺GzmB⁺ over single treatment (FIG. 4 panel G, FIG. 11 panel B). Treatment with anti-IL-35 alone increased CD8⁺ cells and CD8*TNF⁺ cells in the tumors, while the addition of cGAMP augmented this increase (FIG. 4 panel H, FIG. 11 panel C). By contrast, cGAMP plus anti-IL-35 had no additive effect over anti-IL-35 alone on CD8⁺GzmB⁺, CD8⁺IFNγ⁺, CD4⁺CD25⁻, CD4⁺ TNF⁺, CD4⁺IFNγ⁺ or CD4⁺ Foxp3⁺ Treg cells (FIG. 11 panels D-H). Histologically, cGAMP plus anti-IL-35-treated mice showed increased cell death (caspase-3⁺) among keratin⁺ cells and more tumor infiltrating NK1.1⁺ cells (FIG. 12 panels A-C). STING activation is reported to reduce fibrosis (Li et al. 2022 Cellular & Molecular Immunology 19:92-107; Zhao et al. 2019 Gut 68:1827-1837), a prevalent feature of PDAC and this study confirmed that cGAMP reduced expression of fibrosis markers including collagen deposition, Acta2 and Col3a1 mRNA regardless of inclusion of anti-IL-35 (FIG. 12 panels A, D and E).

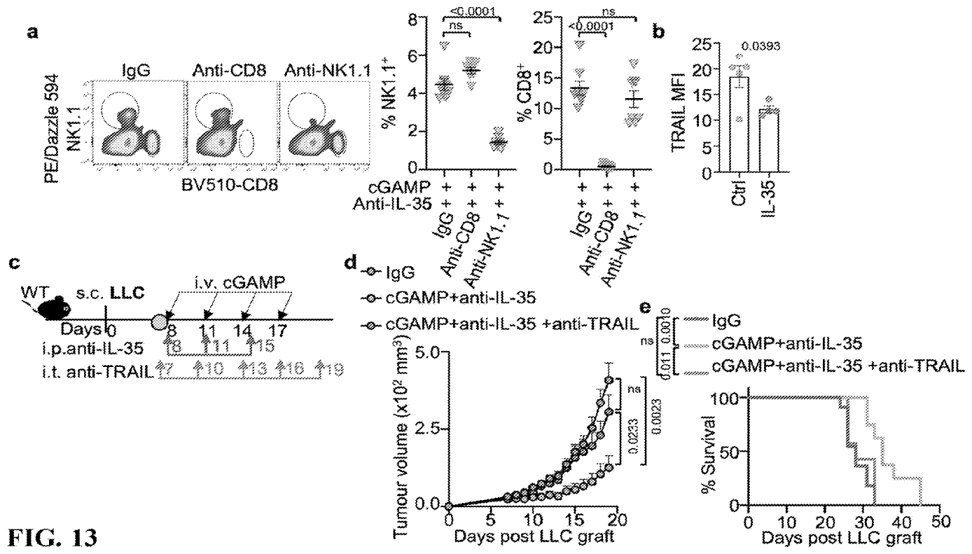

As the combination of cGAMP and anti-IL-35 increased both size of NK1.1⁺ and CD8⁺ subpopulations in orthotopic PDAC, we tested the relevance of these cell types in tumor containment by depleting NK or CD8⁺ T cells (FIG. 4 panel I). The efficiency and specificity of antibody-mediated cell depletion was validated by flow cytometry (FIG. 13 panel A). Tumor reduction following cGAMP plus anti-IL-35 treatment was abrogated when NK1.1⁺ cells, but not CD8⁺ T cells, were depleted (FIG. 4 panel J). This indicates that the anti-tumor effect of the dual therapy is mediated by NK cells. To further explore this link between IL-35 and NK cells, we incubated the NK cell line NK92 MI with IL-35 in vitro, and found that IL-35 treatment reduced NK cytotoxicity against K562, a typical NK target cell (FIG. 4 panel K). To explore possible mechanisms of reduced NK cytotoxicity, the expression of TRAIL, a key mediator of NK killing, was measured, showing that IL-35 reduced TRAIL expression in NK cells (FIG. 13 panel B). Finally, anti-TRAIL neutralizing antibodies abolished tumor reduction following dual therapy (FIG. 13 panels C-E).

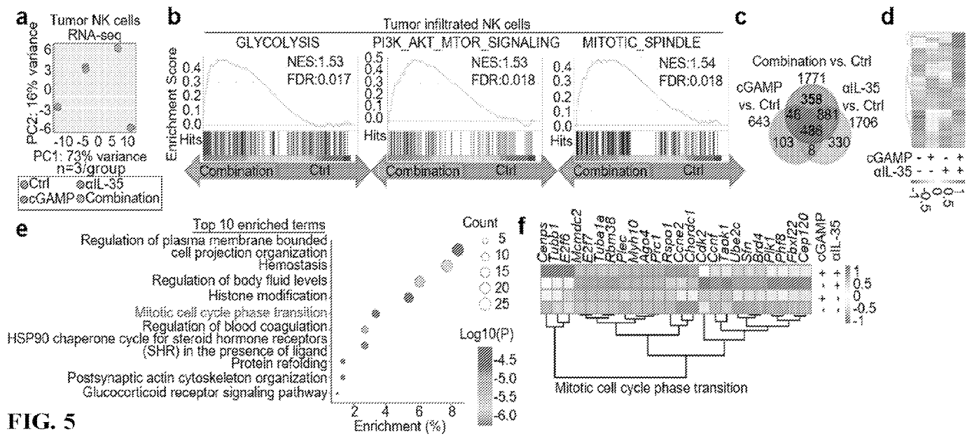

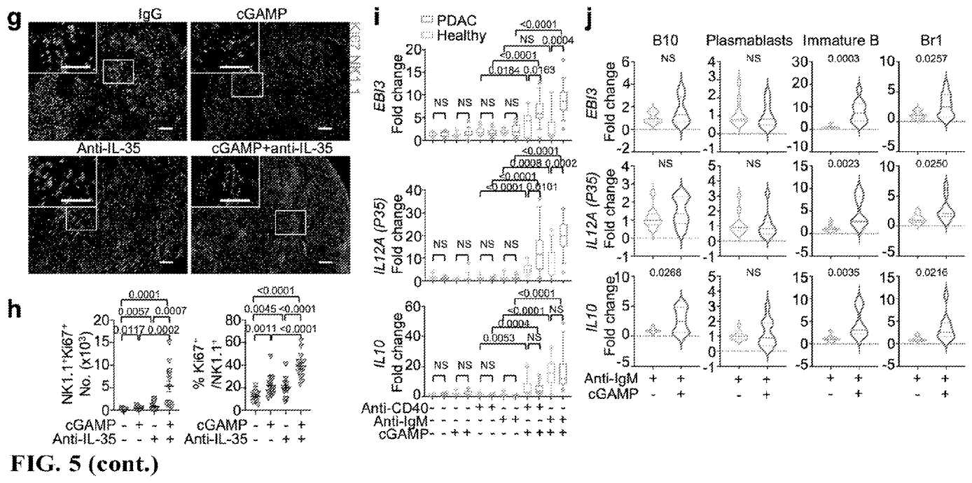

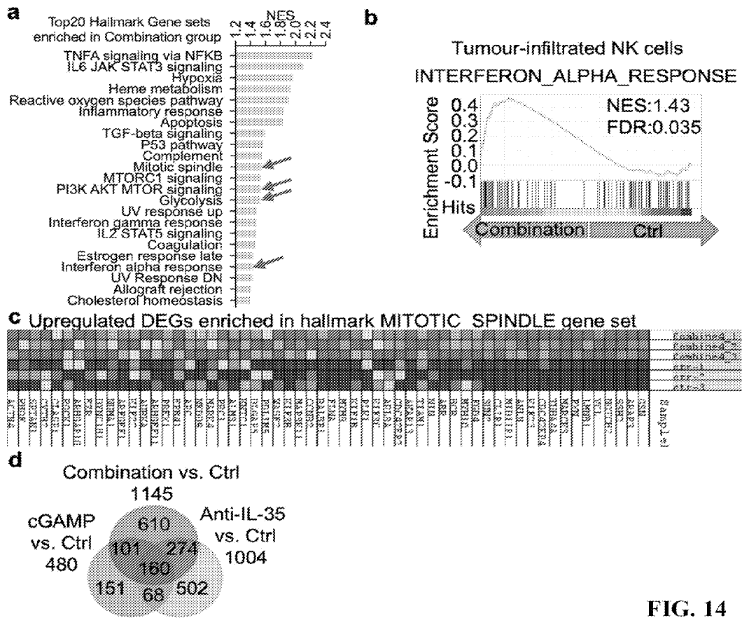

To further understand how intratumoral NK cells are affected by cGAMP and anti-IL-35 treatment in tumors, NK cells from established KPC tumors in mice were sorted and treated with cGAMP and/or anti-IL-35 at day 21 post-tumor cell injection, whereupon RNA from NK cells was extracted and RNAseq analysis was performed. Principal component analysis (PCA) of RNAseq data distinguished NK cells treated with single or combination treatments (FIG. 5 panel A). Gene set enrichment analysis (GSEA) identified top 20 hallmark pathways altered by a combination of cGAMP and anti-IL-35 treatment (FIG. 14 panel A). Among the affected hallmark pathways, known STING associated pathways such as TNFA signaling via NF-kB; IL6, JAK, STAT3 signaling and Interferon alpha response were increased (FIG. 14 panels A and B). The PI3K/AKT/MTOR signaling and glycolysis pathways were also increased. The former is known to drive general cell proliferation and the development, proliferation and effector function of NK cells, whereas the latter is required for NK cell cytotoxicity (FIG. 5 panel B, FIG. 14 panel A). Additionally, the cell cycle associated “Mitotic Spindle” gene set was enriched in the combination treatment group (FIG. 5 panel B, FIG. 14 panel C).

To distinguish the effects of combination versus single treatments, it was noted that the combination treatment vs. control group upregulated 358 (FIG. 5 panels C and D) and downregulated 610 DEGs (FIG. 14 panel D), which were unchanged in either the cGAMP or anti-IL-35 vs. control groups. The top 10 enriched pathways of upregulated DEGs included “mitotic cell cycle phase transition” (FIG. 5 panel E). Genes that were not significantly altered by single treatment but were increased by the combination treatment included Tubb1, which promotes cell proliferation (FIG. 5 panel F). To directly test whether the combination treatment altered NK cell proliferation, this study showed that the combination treatment resulted in robust NK cell proliferation as shown by increased total number and percentage of NK1.1⁺Ki67⁺ cells in the tumor (FIG. 5 panel G, quantitated in FIG. 5 panel H).

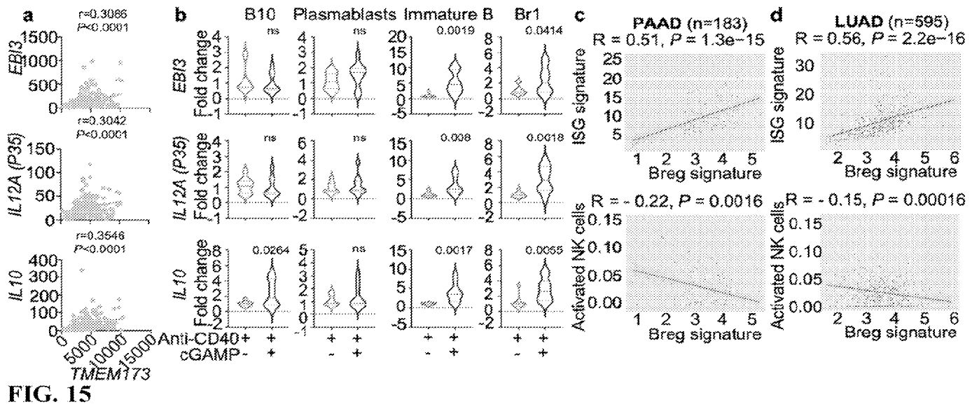

To investigate the link between STING and immunosuppressive cytokines in human pancreatic tumors, the pancreatic adenocarcinoma dataset from The Cancer Genome Atlas (TCGA-PAAD) (Weinstein et al. 2013 Nat Genet 45:1113-1120, incorporated herein by reference) was examined, wherein correlation analysis revealed that human EBI3, IL12A (p35) and IL10 expression positively correlated with TMEM173 transcripts (FIG. 15 panel A). The effect of cGAMP treatment in human PBMC-derived B cells from pancreatic cancer patients and healthy volunteers was directly evaluated. cGAMP treatment enhanced EBI3, IL12A and IL10 expression in activated human B cells from healthy controls. EBI3 and IL12A but not IL10 were further elevated in B cells from pancreatic cancer patients compared to healthy controls (FIG. 5 panel I). To identify the human B cell subsets that produce IL-35 cytokines in response to cGAMP, this study analyzed B10 cells, plasmablasts, immature B cells and Br1 cells (FIG. 7), all have been previously reported as immunosuppressive in auto-immune diseases. Treatment with cGAMP plus either anti-IgM (FIG. 5 panel J) or anti-CD40 (FIG. 15 panel B) selectively enhanced EBI3 and IL12A expression in immature B cells and Br1 cells. Finally, the Breg signature (Bayne et al. 2012 Cancer Cell 21:822-835; Mirlekar et al. 2020 Cancer Immunology Research 8:29-308) correlated positively with the interferon-stimulated gene (ISG) signature (Liu et al. 2019 Nature Medicine 25:95-102), but negatively with activated NK cell signature (Newman et al. 2019 Nat Biotechnol 37:773-782) in the TCGA-PAAD (pancreatic adenocarcinoma) (FIG. 15 panel C) and the LUAD (lung adenocarcinoma) databases (FIG. 15 panel D). Collectively, these data indicate that cGAMP induces immunosuppressive IL-35 preferentially in tumor-bearing patients over healthy controls.

In summary, the findings described herein show that resistance to systemic treatment with STING agonist is attributable partly to the expansion of immunosuppressive B cells that impede NK cells. Whereas others have shown that the STING-triggered IFN response may be involved for anti-tumor NK cell function (Marcus et al. 2018 Immunity 49:754-763), this work provides a counter scenario wherein the systemic delivery of STING agonist suppresses NK cell-mediated anti-tumor responses through B cell-derived IL-35. This is pertinent as systemic delivery of STING agonist exhibits less efficacy than intratumoral delivery of STING agonists. In viral infection, NK cell-dependent cytotoxicity during infection is STING-dependent and EBI3 induced IL-10 in NK cells leads to diminished anti-viral CD8 T cell activity. However, the importance of EBI3 in the regulation of NK cells has not been previously reported in cancer nor have B cells been implicated as major regulators of NK cell during either infection or cancer. This report reveals a new immunoregulatory circuit in cancer where STING activates B regulatory function by inducing IL-35, which then subdues NK cell responses. This negative circuit may be relieved by blocking B cell-specific IL-35 during cGAMP treatment and represents a strategy to control tumors.

### Example 2: Methods

Animals were maintained in a specific pathogen-free facility (12-hour light/dark cycle, temperature: 21-23° C., humidity: 30-70%). All mice, male or female, were used at 6-12 weeks of age, matching age and sex, randomly grouping for experiments. C57BL/6J or wildtype, WT (stock #000664), μMT (stock #002288), Tmem173^{-/-} (stock #017537), Ifnar1^{-/-} (stock #028288), Il10^{-/-} (stock #002251) and p35^{-/-} (stock #002692) strains were from the Jackson Laboratory (Bar Harbor, ME). Irf3^{-/-} mice were provided by T. Taniguchi (University of Tokyo, Tokyo, Japan) and obtained from J. K. Whitmire (University of North Carolina, Chapel Hill, NC). Ebi3^{Tom.L/L} and Foxp3^{Cre-YFP}Ebi3^{Tom.L/L} (T_{reg}(Ebi3^{-/-})) mice were obtained from D. Vignali (University of Pittsburgh, Pittsburgh, PA). Cd19^{cre}Ebi3^{fl/fl} (B(Ebi3^{-/-})) mice were generated by crossing Cd19^{Cre} mice to Ebi3^{fl/fl} mice for two generations to obtain homozygosity at the Ebi3 locus with resulting progeny lacking expression of IL-35 in B cells. Cd19^{cre}Tmem173^{fl/fl} mice or Cd4^{cre}Tmem173^{fl/fl} were generated by crossing Cd19^{Cre} or Cd4^{Cre} mice to Tmem173^{fl/fl} mice (University of Washington School of Medicine, Seattle, WA) with resulting progeny lacking expression of STING in B or T cells. Littermates with WT allele or lacking Cre were used as controls.

Cell lines: The mouse PDAC cell lines KPC4662 and KPC2173 were derived from primary pancreatic tumor isolated from C57BL/6J LSL-Kras^{G12D/+}; LSL-Trp53^{R172H/+}; Pdx1^{Cre/+} (KPC) mice. KPC, LLC, E0771 and B16-ova cells were cultured in complete DMEM (#11995065, Thermo Fisher) containing 10% heat-inactivated fetal bovine serum (FBS) and 1% penicillin-streptomycin (PS) (#15140122, Thermo Fisher). Cells were used at <16 passages and confirmed to be mycoplasma- and endotoxin-free. NK-92 MI NK cells (#CRL-2408, ATCC) were cultured in MEMα (#12561056, Thermo Fisher) media containing 10% FBS, 1% PS, 2 mM L-glutamine (#25030081, Thermo Fisher), 0.1 mM 2-mercaptoethanol, 10 mM HEPES (#15630106, Thermo Fisher) and 0.1 mM non-essential amino acids (#11140050, Thermo Fisher). The erythroleukemic cell line K562 was cultured in complete RPMI-1640 media. All cells were maintained at 37° C. and 5% CO₂.

Orthotopic surgery: For orthotopic intrapancreatic injection of KPC cells, mice were anesthetized using ketamine (100 mg/kg)/xylazine (10 mg/kg; Med-Vet International) cocktail. The depth of anesthesia was confirmed by verifying absence of toe pinch response. An left flank incision was made and KPC cells were injected (28-gauge needle) into the tail of the pancreas. The wound was closed in two layers, with running 5-0 Vicryl RAPIDE sutures (Ethicon) for the body wall and 5-0 PROLENE sutures (Ethicon) for the skin. All animals were given buprenorphine (0.1 mg/kg; Med-Vet International) via subcutaneous (s.c.) injection to alleviate pain once after orthotopic surgery.

Generation of B(p35^{-/-}), B(Il00^{-/-}) and control B(WT) bone marrow chimeric mice: B(p35^{-/-}) and B(Il10^{-/-}) mice with deficiencies in p35 and Il10 restricted to B cells were obtained by reconstituting lethally irradiated recipient mice with a mixture of bone marrow cells from B cell deficient μMT mice (80%) and p35^{-/-}, Il10^{-/-} or WT mice (20%)(FIG. 9 panels A and B). Six weeks later, chimeras were orthotopically inoculated with tumor cells. For validation, B(WT), B(p35^{-/-}) and B(Il10^{-/-}) chimeras were assessed for deletion of p35 and Il10 genes restricted to B cells. Splenic CD19⁺ B and CD3⁺ T cells were purified from B(p35^{-/-}), B(Il10^{-/-}) and corresponding B(WT) chimeras by flow sorting. All sorted populations and remaining non-T/B cells were lysed with genomic DNA extracted using the DNAeasy kit (Qiagen) per manufacturer instructions. The amount of genomic DNA present in these samples was normalized using Actb as the standard, to determine the presence of WT p35 and Il10 alleles in cell subsets.

Tumor growth and treatments: KPC cells (75,000) in 25 μL ice-cold PBS mixed with 25 μL matrigel (354234, Corning) were injected into the pancreas. Mice received 10 μg cGAMP or PBS by intravenous (i.v.) injection, starting on day 8 post tumor cell injection, and repeated every 3 days for a total of 4 times. For dynamic timepoint experiments, tumors were harvested on days 11, 17 and 21. For breast cancer studies, 1×10⁶ E0771 cells were orthotopically inoculated. For lung cancer and melanoma studies, 5×10⁵ LLC or B16-ova cells respectively were s.c. injected into the right flank of mice. B16-ova tumor-bearing mice were vaccinated with 50 μg poly(I:C)-ova on day 6. Tumor growth and survival were monitored. For antibody blockade studies in vivo, mice were intraperitoneally (i.p.) injected with 200 μg anti-IL-35 (clone V1.4C4.22, Shenandoah) twice a week for a total of 3 times starting on day 8 post tumor injection, 200 μg anti-IL-10 (clone JES5-2A5, BioXcell) every three days for four times or IgG isotype control starting on day 8 post tumor injection. For studying the effects of combination therapy of anti-IL-35 with STING agonist and further depleting CD8⁺ T or NK1.1⁺ cells, tumor-bearing mice were treated as described above and received either 200 μg of anti-CD8 (clone YTS 169.4, BioXcell), anti-NK (clone PK136, BioXcell) depleting antibody or IgG isotype control every 3 days, starting on day 7. Tumors were harvested on day 21. For mechanisms of NK cytotoxicity, 50 μg of TRAIL neutralizing antibodies (clone N2B2, Thermo Fisher) were injected intratumorally (i.t.) every three days starting on day 7. Tumor growth and survival were monitored.

Lymphocyte isolation: Single-cell suspensions were prepared from isolated tumors and spleens. Spleens were mechanically disrupted, re-suspended in 1% FBS/PBS and processed following RBC lysis (#00-4300-54, Thermo Fisher). For isolation of tumor-infiltrating lymphocytes, tumor tissue was minced into 1 to 2 mm pieces and digested with collagenase IV (1.25 mg/mL; #LS004188, Worthington), 0.1% soybean trypsin inhibitor (#T9128, Sigma), hyaluronidase (1 mg/mL; #LS002592, Worthington) and DNase I (100 mg/mL; #LS002007, Worthington) in DMEM for 30 min at 37° C. Cell suspensions were passed through a 70 m cell strainer (Falcon) and resuspended in 2% FBS DMEM media. Lymphocytes were isolated from processed tumor tissues by Ficoll density gradient centrifugation. For some experiments, isolation of total CD45⁺ leukocytes by magnetic-activated cell sorting (#130-052-301, MACS Miltenyi Biotec) was performed on the leukocyte-enriched fraction according to manufacturer's protocol, with purity of >90% achieved. Cells were collected in complete RPMI media containing 10% FCS with 1% PS.

Intracellular cytokine staining: For ex vivo stimulation, 10⁶ single cells from tumors or spleens were incubated with PMA (50 ng/mL; #P8139, Sigma) and ionomycin (200 ng/mL; #I-0634, Sigma) in the presence of Golgistop Brefeldin A (1×, BioLegend) in complete RPMI medium for 4 hr at 37° C. Cells were washed and blocked with anti-CD16/CD32 (#101320, Biolegend, 0.1 mg/100,000 cells) for 5 min on ice. After washing, cells were stained with labeled cell surface antibodies on ice for 30 min in FACS buffer (PBS with 1% FBS and 0.05% sodium azide). Viability was assessed using Fixable Viability Ghost Dye Red 780 (#13-0865, Tonbo). After staining, cells were washed, fixed and permeabilized using Cytofix/cytoperm buffer (#554714, BD Biosciences) for 15 min at 4° C. in the dark. Intracellular staining was performed using fluorophore-conjugated cytokine antibodies for 1 hr at 4° C. in the dark. After intracellular staining, cells were washed and resuspended in FACS buffer for acquisition by flow cytometer via FACSDiva (BD, version 8.0.1). Intracellular staining for Foxp3 was performed using a Foxp3 staining kit (#00-5523, eBioscience). Samples were acquired on Attune N×T Cytometer (Thermo Fisher) or LSRII Fortessa (BD Biosciences) with data analyzed using FlowJo version 10.6.2 (BD Biosciences). For human B cells, human BD Fc Block (#422301, Biolegend, 0.1 mg/100,000 cells) was applied for blocking. All antibodies and reagents are listed in Table 1.

Immunoblotting: A million cells were lysed on ice for 30 min using RIPA lysis and extraction buffer (Thermo Fisher), supplemented with protease inhibitor cocktail (Roche). The lysate was centrifuged at 16,000×g at 4° C. for 10 min. Clarified supernatants were loaded into 4-12% NuPAGE Bis-Tris gels (Thermo Fisher), transferred to PVDF membranes (Bio-Rad) and blocked for 1 hr with 5% non-fat milk in TBST buffer. Membranes were incubated with primary antibodies in blocking buffer overnight at 4° C. After washing five times, membranes were incubated with secondary antibodies for 1 hr at room temperature (RT). Proteins were detected using Femto Chemiluminescent reagent (Thermo Scientific). Antibodies are listed in Table 1.

Enzyme-linked immunosorbent assay (ELISA): Tumor and spleen homogenates were prepared using extraction buffer (100 mM Tris, pH 7.4, 150 mM NaCl, 1 mM EGTA, 1 mM EDTA, 1% Triton X-100, 0.5% Sodium deoxycholate, Phosphatase inhibitor cocktail, Protease inhibitor cocktail, PMSF). Samples were incubated at 4° C. for 1 hr on the orbital shaker and supernatants were collected by centrifugation at 9000×rpm for 10 min at 4° C. All samples were stored at −80° C. The concentrations of IL-35 and IL-10 were measured using LEGEND MAX™ Mouse IL-35 Heterodimer ELISA Kit (#440507) and ELISA MAX™ Standard Set Mouse IL-10 kit (#431414) according to manufacturer's protocols (Biolegend).

Realtime PCR analysis for gene expression: RNA was prepared from treated cells using RNeasy Mini Kit (#74106, Qiagen) or TRIzol extraction (#15596026, Thermo Fisher). Complementary DNA was generated using a High Capacity cDNA-RT Kit (Invitrogen). Quantitative PCR analyses were performed using universal SYBR green super-mix reagent (Bio-Rad) and QuantStudio 6 Flex real-time PCR system (Thermo Fisher). Results were normalized to Actb expression. Gene expression was determined by the ΔΔCt method (2-ΔΔCt). Primer sequences are listed in Table 2.

Cell stimulation assays: Mouse splenic B or T cells were sorted using negative selection (#130-090-862 and #130-095-130, Miltenyi Biotec) from spleens of tumor-bearing or healthy mice. Total B cells were seeded in 96-well flat-bottom plates and stimulated with 5 μg/mL anti-IgM. T cells were seeded and stimulated with 5 μg/mL anti-CD3 and 2 μg/mL anti-CD28. Cells were treated simultaneously with or without 5 μg/mL cGAMP. For experiments in FIG. 2 panel C, B cells were stimulated by 5 μg/mL anti-IgM, 2 μg/mL anti-CD40 or 2 μg/mL LPS with or without 5 μg/mL cGAMP for 24 hr. For experiments in FIG. 7 panels H-J, B cells were stimulated by 5 μg/mL anti-IgM with increasing doses of cGAMP (0, 1, 5, 10 μg/mL) for 24 hr. For experiments in FIG. 2 panel D and FIG. 7 panel K., B cells were stimulated by 5 μg/mL anti-IgM with 5 μg/mL DMXAA (Conlon et al. 2013 J Immunol 190:5216-5225), ADU-S100 (Ramanjulu et al. 2018 Nature 564:439-443), diABZI (Corrales et al. 2015 Cell Reports 11:1018-1030) or MSA-2 (Pan et al. 2020 Science 369:eaba6098) for 24 hr. Cells were processed for flow cytometry. For RNA evaluation, mouse B cells were first stimulated using 5 μg/mL anti-IgM and cGAMP for 12 hr and then processed for analysis.

Primary NK cell treatment: Tumoral NK cells were sorted from KPC bearing mice by FACS cell sorter. NK cells (3×10⁵) were seeded in 24-well plates and stimulated with 100 ng/mL mouse IL-35 (#CHI-MF-11135, Chimerigen Labs). Cells were analyzed by FACS staining.

MTT viability assay: For the MTT assay, 10³ KPC4662 cells were seeded, stimulated with cGAMP for 24 hr and 250 μg/mL MTT was added 4 hr before the end point. The plate was centrifuged and supernatant was discarded. DMSO (150 μL) was added to dissolve the pellets. The absorbance was read at OD590 nm.

NK cell-mediated cytotoxicity assay: After incubation with 100 ng/mL IL-35 for 48 hr, NK-92 MI cells were subjected to Ficoll density gradient centrifugation. Viable cells were washed and counted to add target K562 and effector NK-92 MI cells in effector/target ratios of 5:1, 2.5:1, 0.5:1 and 0.25:1 in 96-well U-bottom plates. After 3 hr of co-culture, Lactate Dehydrogenase (LDH) assay was performed according to manufacturer's instructions (#11644793001, SigmaAldrich).

Human samples: Blood samples were collected from 15 patients with pancreatic cancer and 15 healthy controls. PBMCs were obtained by separating whole blood samples via density gradient centrifugation (Lymphoprep, Axis-shield). Briefly, collected blood samples in yellow top ACD tubes (~20 mL each) were diluted with an equal volume of 0.9% NaCl. Carefully, 20 mL of diluted blood were layered on top of 10 mL Lymphoprep in a 50 mL centrifuge tube to avoid mixing blood and separation fluid and then, centrifuged at 800×g for 20 min at 18° C. Afterward, the mononuclear cells were removed from the sample/medium interface using a Pasteur pipette. The cells were collected by centrifugation at 250×g for 5 min and washed three times with RPMI-1640 media. Human CD19⁺ B cells of pancreatic cancer patients and healthy donors were sorted from PBMCs and stimulated using 5 μg/mL anti-IgM or 2 μg/mL anti-CD40 with or without 5 μg/mL cGAMP for 24 hr. Cells were collected for qPCR assessment of mRNA expression.

Chromatin immunoprecipitation assays: Briefly, splenic B cells isolated from WT and Tmem173^{-/-} mice were stimulated with anti-IgM and cGAMP for 24 hr. After activation, cells were harvested and cross-linked with 37% (W/V) formaldehyde at a final concentration of 1.42% for 15 min at RT. Formaldehyde quenching was done with 125 mM glycine for 5 min at RT. Cell lysis was performed using immunoprecipitation (IP) buffer (150 mM NaCl, 50 mM Tris-HCl pH 7.5, 5 mM EDTA, 0.5% V/V NP-40 and 1% V/V Triton X-100, 0.1 M PMSF, phosphatase inhibitor). Chromatin was sheared into 500-1000 base pair fragments with four rounds of 15 sec sonication pulses with a 2 min rest between each round using a Diagenode Bioruptor. Sheared chromatin was then subjected to IP with IRF3 (Biolegend; 12A4A35; 1:50 diluted) or isotype control antibody (Biolegend; 27-35), followed by overnight incubation with rotation. DNA-protein complexes were immunoprecipitated using protein A-agarose beads (Millipore:Sigma, 17-5280-01) at 4° C. for 1 hr with rotation, washed with IP buffer without inhibitors and subjected to DNA isolation, performed using 10% (W/V) chelex-100 (Bio-Rad; 142-1253) slurry followed by precipitation with 70% ethanol. Purified DNA was used to perform real-time PCR with SYBR green master-mix in 10 μL reaction volumes (2.5 μL DNA template, 0.3 μL of 10 μM primer pair, 5 μL master-mix and 2.2 μL PCR grade water). Relative occupancy of the immunoprecipitated factor at the target locus was estimated by using 2\hat{ }(^{CTControl-CTsample}) methods. Relative enrichment of upstream binding at transcriptional start site (TSS) are scaled to ChIP with control isotype antibody and input. The primers are listed in Table 2.

RNA sequencing (RNAseq): For RNAseq analysis, total RNA was extracted from splenic B cells or sorted tumoral NK cells using the RNeasy Mini Kit (Qiagen, 74106). A total amount of 1 μg RNA per sample was used to generate sequencing libraries using NEBNext® Ultra™ RNA Library Prep Kit from Illumina® (New England Biolabs, Inc., NEB, MA) according to manufacturer's recommendations. Briefly, mRNA was purified from total RNA using poly-T oligo-attached magnetic beads. First-strand cDNA was synthesized using random hexamer primer and M-MuLV Reverse Transcriptase (RNase H−). Second strand cDNA synthesis was performed using DNA Polymerase I and RNase H. Remaining overhangs were converted into blunt ends via exonuclease/polymerase activities. After adenylation of 3′ ends of DNA fragments, NEBNext Adaptor with hairpin loop structures were ligated for hybridization. To select cDNA fragments of preferable lengths of 150-200base pairs, the library fragments were purified with AMPure XP system (Beckman Coulter, Beverly, MA). Then 3 μL USER Enzyme (NEB, MA) was incubated with size-selected, adaptor-ligated cDNA at 37° C. for 15 min followed by 5 min at 95° C. PCR was performed with Phusion High-Fidelity DNA polymerase, Universal PCR primers and Index (X) Primers. Finally, PCR products were purified (AMPure XP system) and library quality was assessed on the Agilent Bioanalyzer 2100 system. Clean data were obtained by removing reads from raw data containing adapter or poly-N sequences and reads with low quality using fastp⁵⁸ with default parameters. All the downstream analyses were based on clean data with high quality. Paired-end clean reads were aligned to the GRCm38/mm10 genome assembly of the Mus musculus reference genome using the Spliced Transcripts Alignment to a Reference (STAR) software. FeatureCounts were used to calculate read counts for each gene based on the aligned files. The count matrix for all samples was used to perform differential expression analysis between two conditions or groups (three biological replicates per condition) using DESeq2 R package. The resulting P-values were adjusted using the Benjamini and Hochberg's approach for controlling the False Discovery Rate (FDR). Genes with an adjusted P<0.05 found by DESeq2 were assigned as differentially expressed. To obtain gene expression value matrix for each sample, FeatureCounts was also used to convert the reads counts to RPKM (reads per kilobase of exon model per million mapped reads) values. R package pheatmap (CRAN.R-project.org/package=pheatmap) was used to generate heatmaps based on this RPKM matrix. KEGG pathway enrichment analysis for DEGs was performed by the R package clusterProfiler.

Gene Set Enrichment Analysis (GSEA): GSEA was performed using the GSEA 4.1.0 Java desktop application downloaded from GSEA (gsea-msigdb.org/gsea/index.jsp). For this study, the hallmark gene sets (MSigDB version 7.2) and Kyoto Encyclopedia of Genes and Genomes gene set collections (MSigDB version 7.2) were used in the analysis. GSEA was performed using the gene_set permutation type with the number of permutations set at 1000. FDR cutoff of P<0.05 was used to identify significantly enriched gene sets.

The Cancer Genome Atlas analyses: Gene expression and clinical data for Pancreatic adenocarcinoma (PAAD) were downloaded from TCGA. For signatures, differential expression analysis was conducted using DESeq2. The Breg gene signature value was calculated for each sample with cancer types of PAAD or LUAD using the geometric mean. The ISG gene signature was calculated in the same way (geometric mean for each sample with cancer types PAAD or LUAD). The activated NK cell signature was from CIBERSORT (version 1.05). Spearman correlations were used to compare the Breg gene signature with the ISG gene signature or the activated NK cell signature and plotted using ggplot2.

Immunohistochemistry and microscopy: Tumor blocks were fixed with 10% formalin overnight, transferred into 70% ethanol, followed by paraffin embedding and cutting into 5 M sections. Prior to immunostaining, tissues were deparaffinized, rehydrated and subjected to antigen retrieval (Abcam, #ab93678). The vendor's instructions for Sirius red staining (Abcam, #ab150681) were followed. The sections were blocked with 10% goat serum. The anti-Ki67 (ThermoFisher, #11-5698-82, 1:200), anti-Keratin (Sigma-Aldrich, #MABT329, 1:200), anti-NK1.1 (Biolegend, #108747, 1:200) or anti-Cleaved Caspase-3 (cell signaling, #9661, 1:400) antibodies were used to stain sections. Tissue sections were incubated with primary antibodies for 2 hr at RT. Fluorescently labelled secondary antibodies were incubated for 30 min at RT. Images were acquired with a Keyence BZ-X710 microscope and analyzed in a blinded fashion with image J software for automated counting.

Statistical analyses: Experiments were independently repeated two to five times. For in vivo experiments, mice were assigned to groups randomly and maintained mixed among cages when possible. Data were analyzed using Prism version 9.0 software (GraphPad) and presented as mean values±s.e.m. Unpaired two-tailed Student's t tests, one-way ANOVA, two-way ANOVA and Log-rank (Mantel Cox) test were used, where P<0.05 was considered statistically significant.

TABLE 1
Non-limiting examples of antibodies and reagents.

| | Source | Identifier(Clone) |
|---|---|---|
| Antibody(Anti-mouse) | | |
| CD19 | Biolegend | 6D5 |
| CD8 | Biolegend | 53-6.7 |
| IL-35 p35 | R & D systems | 27537 |
| IL-35 EBI3 | R & D systems | 355022 |
| NK1.1 | Biolegend | PK136 |
| TNF | Biolegend | MP6-XT22 |
| IL-10 | Biolegend | JES5-16E3 |
| CD4 | Biolegend | GK1.5 |
| CD45 | Biolegend | 30-F11 |
| CD25 | Biolegend | PC61 |
| CD11b | Biolegend | M1/70 |
| Foxp3 | Biolegend | MF-14 |
| IFNγ | Biolegend | XMG1.2 |
| TRAIL | Biolegend | N2B2 |
| CD5 | Biolegend | 53-7.3 |
| CD21 | Biolegend | 7E9 |
| CD3 | Biolegend | 17A2 |
| Granzyme B | Biolegend | QA18A28 |
| CD1d | Biolegend | 1B1 |
| IgM | Biolegend | RMM-1 |
| IgD | Biolegend | 11-26c.2a |
| CD138 | Biolegend | 281-2 |
| Ki-67 | Thermo Fisher | SolA15 |
| Cleaved Caspase-3 | cell signaling | 9661S |
| Keratin | Sigma-Aldrich | TROMA-1 |
| PE/Dazzle ™ 594 anti-mouse NK-1.1 | Biolegend | PK136 |
| IRF3 (D83B9) Rabbit mAb | Cell Signaling | 4302-CS |
| | Technology | |
| Phospho-IRF3 (Ser396) (4D4G) | Cell Signaling | 4947-CS |
| Rabbit mAb | Technology | |
| NF-κB p65 (D14E12) XP ® | Cell Signaling | 8242-CS |
| Rabbit mAb | Technology | |
| Phospho-NF-κB p65 (Ser536) | Cell Signaling | 3033-CS |
| (93H1) Rabbit mAb | Technology | |
| STING (D2P2F) Rabbit mAb | Cell Signaling | 13647-CS |
| | Technology | |
| Actin (C-11) | santa cruz | sc-1615 |
| αIL-35 | Shenandoah Inc | V1.4C4.22 |
| αIL-10 | BioXCell | JES5-2A5 |
| αNK1.1 | BioXCell | PK136 |
| αCD8α | BioXCell | YTS 169.4 |
| αTRAIL | Thermo Fisher | N2B2 |
| IgG | BioXCell | MPC-11 |
| Purified aCD3 | Thermo Fisher | 16-0031-85 |
| Purified aCD28 | Thermo Fisher | 16-0281-85 |
| F(ab′)2-Goat αMouse IgM | Thermo Fisher | 16-5092-85 |
| CD40 Monoclonal Antibody (HM40-3) | Thermo Fisher | 14-0402-86 |
| TruStain FcX ™ (anti-mouse CD16/32) | Biolegend | 93 |
| Antibody(Anti-human) | | |
| CD19 | Biolegend | HIB19 |
| CD24 | Biolegend | ML5 |
| CD38 | Biolegend | HB-7 |
| CD27 | Biolegend | O323 |
| CD71 | Biolegend | CY1G4 |
| CD25 | Biolegend | BC96 |
| Human TruStain FcX ™ (Fc | Biolegend | 422301 |
| Receptor Blocking Solution) | | |
| αCD40 | Biolegend | 334302 |
| F(ab′)2-Goat anti-Human IgG, IgM (H + L) | ThermoFisher | 16-5099-85 |
| Reagents | | |
| Normal Goat Serum | Jackson | 005-000-121 |
| | ImmunoResearch | |
| ProLong ™ Gold Antifade Mountant | Thermo Fisher | P36930 |
| DAPI | ThermoFisher | D1306 |
| Ghost Dye ™ Red 780 | Tonbo | 13-0865 |
| LEGEND MAX ™ Mouse IL-35 Heterodimer | Biolegend | 440507 |
| ELISA Kit | | |
| ELISA MAX ™ Deluxe Set Mouse IL-10 | Biolegend | 431414 |
| LEGEND MAX ™ Mouse IFN-β ELISA Kit | Biolegend | 439407 |
| 3′3′-cGAMP | Invivogen | tlrl-nacga |
| DMXAA | Invivogen | tlrl-dmx |
| diABZI STING agonist (Compound 3) | selleckchem | S8796 |
| Recombinant mouse IL-35 | Chimerigen Labs | CHI-MF-11135 |
| Recombinant human IL-35 | Chimerigen Labs | CHI-HF-21035 |
| ADU-S100 | Invivogen | tlrl-nacda2r-01 |
| MSA-2 | selleckchem | S9681 |
| MTT assay kit | promega | G4000 |
| Lipopolysaccharides | Sigma | L2387-10MG |
| Phorbol 12-myristate 13-acetate (PMA) | Sigma | P8139 |
| Ionomycin | Sigma | I0634-1MG |
| Brefeldin A Solution (1,000X) | Biolegend | 420601 |
| Picro Sirius Red Stain Kit (Connective | abcam | ab150681 |
| Tissue Stain) | | |
| Cytofix/Cytoperm Fixation/Permeabilization | BD Biosciences | 554714 |
| Solution Kit | | |
| Foxp3/Transcription Factor Staining Buffer Set | eBioscience | 00-5523-00 |
| TRIzol | Thermo Fisher | 15596026 |
| Ficoll Paque Premium | Millipore Sigma | 17-5442-02 |
| Lactate Dehydrogenase (LDH) assay | Millipore Sigma | 11644793001 |
| Hyaluronidase | Worthington | LS002592 |
| Soybean trypsin inhibitor | Sigma | T9128 |
| Collagenase IV | Worthington | LS004188 |
| DNase I | Worthington | LS002007 |
| 10X RBC Lysis Buffer | Thermo Fisher | 00-4300-54 |
| Matrigel | Corning | 354234 |
| Pan T Cell Isolation Kit II, mouse | Miltenyi Biotec | 130-095-130 |
| B Cell Isolation Kit, mouse | Miltenyi Biotec | 130-090-862 |
| EasySep ™ Human B Cell Isolation Kit | Stemcell | 17954 |
| EasySep Mouse APC Positive Selection Kit II | Stemcell | 17667 |
| EasySep Mouse NK Cell Isolation Kit | Stemcell | 19855 |
| CD45 MicroBeads, mouse | MACS Miltenyi Biotec | 130-052-301 |
| LD Columns | Miltenyi Biotec | 130-042-901 |
| CFSE | Biolegend | 423801 |
| CellTrace Violet | ThermoFisher | C34557 |
| Reagents | Source | Identifier |
| Normal Goat Serum | Jackson | 005-000-121 |
| | ImmunoResearch | |
| ProLong ™ Gold Antifade Mountant | Thermo Fisher | P36930 |
| DAPI | ThermoFisher | D1306 |
| Ghost Dye ™ Red 780 | Tonbo | 13-0865 |
| LEGEND MAX ™ Mouse IL-35 Heterodimer | Biolegend | 440507 |
| ELISA Kit | | |
| ELISA MAX ™ Deluxe Set Mouse IL-10 | Biolegend | 431414 |
| LEGEND MAX ™ Mouse IFN-β ELISA Kit | Biolegend | 439407 |
| 3′3′-cGAMP | Invivogen | tlrl-nacga |
| DMXAA | Invivogen | tlrl-dmx |
| diABZI STING agonist (Compound 3) | selleckchem | S8796 |
| Recombinant mouse IL-35 | Chimerigen Labs | CHI-MF-11135 |
| Recombinant human IL-35 | Chimerigen Labs | CHI-HF-21035 |
| ADU-S100 | Invivogen | tlrl-nacda2r-01 |
| MSA-2 | selleckchem | S9681 |
| MTT assay kit | promega | G4000 |
| Lipopolysaccharides | Sigma | L2387-10MG |
| Phorbol 12-myristate 13-acetate (PMA) | Sigma | P8139 |
| Ionomycin | Sigma | I0634-1MG |
| Brefeldin A Solution (1,000X) | Biolegend | 420601 |
| Picro Sirius Red Stain Kit (Connective | abcam | ab150681 |
| Tissue Stain) | | |
| Cytofix/Cytoperm Fixation/Permeabilization | BD Biosciences | 554714 |
| Solution Kit | | |
| Foxp3/Transcription Factor Staining Buffer Set | eBioscience | 00-5523-00 |
| TRIzol | Thermo Fisher | 15596026 |
| Ficoll Paque Premium | Millipore Sigma | 17-5442-02 |
| Lactate Dehydrogenase (LDH) assay | Millipore Sigma | 11644793001 |
| Hyaluronidase | Worthington | LS002592 |
| Soybean trypsin inhibitor | Sigma | T9128 |
| Collagenase IV | Worthington | LS004188 |
| DNase I | Worthington | LS002007 |
| 10X RBC Lysis Buffer | Thermo Fisher | 00-4300-54 |
| Matrigel | Corning | 354234 |
| Pan T Cell Isolation Kit II, mouse | Miltenyi Biotec | 130-095-130 |
| B Cell Isolation Kit, mouse | Miltenyi Biotec | 130-090-862 |
| EasySep ™ Human B Cell Isolation Kit | Stemcell | 17954 |
| EasySep Mouse APC Positive Selection Kit II | Stemcell | 17667 |
| EasySep Mouse NK Cell Isolation Kit | Stemcell | 19855 |
| CD45 MicroBeads, mouse | MACS Miltenyi Biotec | 130-052-301 |
| LD Columns | Miltenyi Biotec | 130-042-901 |
| CFSE | Biolegend | 423801 |
| CellTrace Violet | ThermoFisher | C34557 |

TABLE 2
List of primers.

| mouse qPCR primers | | | | |
|---|---|---|---|---|
| | Forward | SEQ | Reverse | SEQ |
| Primer | (5′-3′) | ID NO: | (5′-3′) | ID NO: |
| Ifnb1 | GCCTTTGCCATCCAAG | 1 | ACACTGTCTGCTGGTG | 2 |
| | AGATGC | | GAGTTC | |
| Ifna4 | GCAATGACCTCCATCA | 3 | GTGGAAGTATGTCCTC | 4 |
| | GCAGCT | | ACAGCC | |
| Il10 | CGGGAAGACAATAAC | 5 | CGGTTAGCAGTATGTT | 6 |
| | TGCACCC | | GTCCAGC | |
| Il12a | ACGCAGCACTTCAGA | 7 | CGCAGAGTCTCGCCAT | 8 |
| | ATCAC | | TATG | |
| Ebi3 | CTCTCAAGTACCGACT | 9 | CTGAGCTGACACCTG | 10 |
| | CCGCTA | | GATGCAA | |
| Col3a1 | GACCAAAAGGTGATG | 11 | CAAGACCTCGTGCTCC | 12 |
| | CTGGACAG | | AGTTAG | |
| Acta2(α- | TGCTGACAGAGGC | 13 | CAGTTGTACGTCC | 14 |
| SMA) | ACCACTGAA | | AGAGGCATAG | |
| Actb | CATTGCTGACAGGATG | 15 | TGCTGGAAGGTGGAC | 16 |
| | CAGAAGG | | AGTGAGG | |
| ChIP- qPCR primers | | | | |
| | Forward | SEQ | Reverse | SEQ |
| Primer | (5′-3′) | ID NO: | (5′-3′) | ID NO: |
| Ebi3-800 | ACAGATGACTCTCTCA | 17 | AGGGTCTAGGGCCTT | 18 |
| | CTCTTCT | | GAGTT | |
| Ebi3-1000 | GATGGGGCAAAGAGG | 19 | CCCCTTCCTGGGGGTA | 20 |
| | GCTAAA | | TAAA | |
| p35-700 | TGGACTCACCTGGATG | 21 | TTGCCCAGGAGGTTAC | 22 |
| | GCA | | AATGAA | |
| p35-1500 | ACTCATCACTGGCTCA | 23 | CACGCGTACTTGCTCT | 24 |
| | GCATC | | CTGT | |
| Il10-500 | AGAAAATCAGCCCTCT | 25 | CATTCACGCACTCCCA | 26 |
| | CGGG | | GGTC | |
| Il10-1400 | CATCAGCTCAGAGAG | 27 | GATCAGGATCAAGGG | 28 |
| | GCAGTT | | GGTCT | |
| mouse genotyping PCR primers for | | | | |
| bone marrow chimeric mice | | | | |
| | Forward | SEQ | Reverse | SEQ |
| Primer | (5′-3′) | ID NO: | (5′-3′) | ID NO: |
| Il10 | CGGGAAGACAATAAC | 29 | CGGTTAGCAGTATGTT | 30 |
| | TGCACCC | | GTCCAGC | |
| Il12a | ACGAGAGTTGCCTGGC | 31 | CCTCATAGATGCTACC | 32 |
| | TACTAG | | AAGGCAC | |
| Actb | GGCTGTATTCCCCTCC | 33 | CCAGTTGGTAACAAT | 34 |
| | ATCG | | GCCATGT | |
| human qPCR primers | | | | |
| | Forward | SEQ | Reverse | SEQ |
| Primer | (5′-3′) | ID NO: | (5′-3′) | ID NO: |
| IL10 | GCCTAACATGCTTCGA | 35 | TGATGTCTGGGTCTTG | 36 |
| | GATC | | GTTC | |
| IL12A | GATGAGCTGATGCAG | 37 | AGTCCTCCACCTCGTT | 38 |
| | GCC | | GTCCGTGA | |
| EBI3 | GCAGACGCCAACGTC | 39 | CCAGTCACTCAGTTCC | 40 |
| | CAC | | CCGT | |
| HPRT | CCTGCTGGATTACATC | 41 | TCCAACACTTCGTGGG | 42 |
| | AAAGCACTG | | GTCCT | |

The foregoing is illustrative of the present invention, and is not to be construed as limiting thereof. The invention is defined by the following claims, with equivalents of the claims to be included therein.

1. A method of treating cancer in a subject, comprising administering to the subject an effective amount of a Stimulator of Interferon Genes (STING) agonist in combination with an interleukin (IL)-35 inhibitor, thereby treating the cancer.

2. A method of enhancing an immune response in a subject having, suspected to have, and/or at risk of cancer, comprising administering to the subject an effective amount of a Stimulator of Interferon Genes (STING) agonist in combination with an interleukin (IL)-35 inhibitor, thereby enhancing the immune response to the cancer.

3. (canceled)

4. The method of claim 1, wherein the cancer comprises a tumor microenvironment, optionally wherein the tumor microenvironment is immunosuppressive.

5. (canceled)

6. The method of claim 1, wherein the subject has previously received an immunotherapy.

7. The method of claim 6, wherein the cancer in the subject was resistant to the previous immunotherapy.

8. (canceled)

9. The method of claim 1, wherein the STING agonist is cGAMP, DMXAA, diABZI, ADU-S100, G10 or MSA-2.

10. The method of claim 1, wherein the IL-35 inhibitor is an anti-IL-35 antibody or an antigen-binding fragment thereof.

11. The method of claim 1, wherein the cancer is a solid tumor.

12. The method of claim 11, wherein the cancer is pancreatic cancer, breast cancer, lung cancer and/or melanoma.

13. The method of claim 1, wherein the administering comprises one or more administrations of the STING agonist and inhibitor of IL-35 every 1, 2, 3, 4, 5, 6, 7, 8, 9, 10 or more days.

14. The method of claim 13, wherein the one or more administrations comprises administering the STING agonist about every 3 days for 4 repeats and/or administering the IL35 inhibitor about twice a week for 3 repeats.

15. (canceled)

16. The method of claim 1, wherein an effective amount comprises a concentration of about 1 ug to about 100 ug STING agonist and/or a concentration of about 1 ug to about 500 ug inhibitor of IL-35.

17. The method of claim 16, wherein an effective amount comprises a concentration of about 10 ug STING agonist and/or a concentration of about 200 ug inhibitor of IL-35.

18. The method of claim 1, wherein the STING agonist and inhibitor of IL-35 are concurrently co-administered, wherein the STING agonist and inhibitor of IL-35 are administered as a single composition, wherein the STING agonist is administered prior to the IL-35 inhibitor, wherein the STING agonist is administered after the IL-35 inhibitor, or any combination thereof.

19-20. (canceled)

21. The method of any one of claim 1, further comprising co-delivering a therapeutic agent to the subject.

22. (canceled)

23. The method of claim 21, wherein the therapeutic agent is an immunotherapy agent, radiation therapy, and/or chemotherapy.

24. (canceled)

25. A composition or combination comprising a STING agonist and IL-35 inhibitor, optionally further comprising a pharmaceutical carrier, diluent, and/or adjuvant.

26. (canceled)

27. The composition or combination of claim 25, wherein the STING agonist is G10, cGAMP, DMXAA, diABZI, ADU-S100, or MSA-2.

28. The composition or combination of claim 25, wherein the IL-35 inhibitor is an anti-IL-35 antibody or an antigen-binding fragment thereof.

29. (canceled)

30. A kit comprising the composition or combination of claim 25, and optional instructions for the use thereof.
